(19) United States
(12) Patent Application Publication
(10) Pub. No.: US 2022/0177426 A1
(43) Pub. Date: Jun. 9, 2022

(54) PROCESS FOR PREPARING ALPHA-CARBOXAMIDE PYRROLIDINE DERIVATIVES

(71) Applicant: Biogen MA Inc., Cambridge, MA (US)

(72) Inventors: Donald G. Walker, Littleton, MA (US); Dino Alberico, Mississuga (CA); Joshua Clayton, Stoney Creek (CA); Chandrasekhar Navuluri, Mississauga (CA)

(21) Appl. No.: 17/602,585

(22) PCT Filed: Apr. 9, 2020
(86) PCT No.: PCT/US2020/027459

§ 371 Date: Oct. 8, 2021

Related U.S. Application Data
(60) Provisional application No. 62/831,962, filed on Apr. 10, 2019.

(51) Int. Cl. C07D 207/16; C07D 207/28; C07D 263/18; C07C 269/06; C07C 51/60

(52) U.S. Cl. CPC C07D 207/16; C07D 207/28; C07C 51/60; C07C 269/06; C07D 263/18

(57) ABSTRACT

Disclosed are processes for preparing α-carboxamide pyrrolidine derivatives, in particular (2S,5R)-5-(4-((2-fluorobenzyl)oxy)phenyl)pyrrolidine-2-carboxamide, and intermediates for use in said processes along with processes for preparing said intermediates.

### RELATED APPLICATIONS

This application claims the benefit of U.S. Provisional Application No. 62/831,962, filed Apr. 10, 2019, the contents of which are incorporated herein by reference in their entirety.

### BACKGROUND

(2S,5R)-5-(4-((2-fluorobenzyl)oxy)phenyl)pyrrolidine-2-carboxamide:

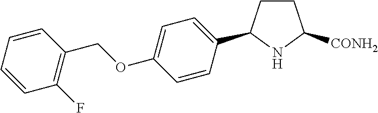 [SMILES: NC(=O)[C@@H]1CC[C@H](c2ccc(OCc3ccccc3F)cc2)N1]

is described in U.S. Pat. No. 7,655,693 as having utility in the treatment of diseases and conditions mediated by modulation of use-dependent voltage-gated sodium channels. Certain synthetic methods to prepare (2S,5R)-5-(4-((2-fluorobenzyl)oxy)phenyl)pyrrolidine-2-carboxamide are described in U.S. Pat. Nos. 7,655,693 and 8,759,542. The contents of each of these patents are incorporated by reference in their entirety.

However, there is a need for the development of alternative processes for the preparation of such α-carboxamide pyrrolidine derivatives, which are capable of practical application to large scale manufacture.

### SUMMARY

The present disclosure provides processes for preparing a compound of formula (I)

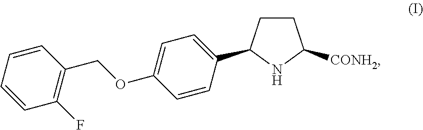 [SMILES: NC(=O)[C@@H]1CC[C@H](c2ccc(OCc3ccccc3F)cc2)N1]

or a pharmaceutically acceptable salt thereof, comprising reacting a compound of formula (III) with a compound of formula (IV) in the presence of a base, thereby producing a compound of formula (V):

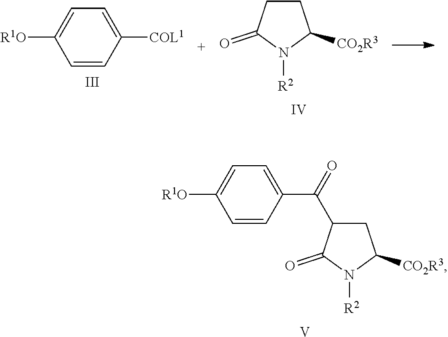

wherein L¹ is a leaving group (such as a halide, e.g., Br or Cl); R¹ is an oxygen-protecting group (such as allyl, benzyl, benzoyl, methoxymethyl, tetrahydropyranyl, tert-butyl, acetyl, silicon-containing protecting group). In certain preferred embodiments, R¹ is benzyl. R² is a resonance-accepting nitrogen-protecting group, such as nitrogen-protecting group selected from: tert-butyloxycarbonyl (Boc); 9-fluorenylmethyloxycarbonyl (Fmoc); acetyl (Ac); benzoyl (Bz); carbamates; tosyl (Ts); a sulfonamide selected from Nosyl and Nps and trifluoroacetyl. In certain preferred embodiments, R² is tert-butyloxycarbonyl (Boc). In certain embodiments, R¹ is C₁₋₆ alkyl, C₂₋₆ alkenyl, or C₂₋₆ alkynyl, most preferably methyl. In certain embodiments, the process is for preparing a compound of formula (V).

The present disclosure further provides processes for preparing a compound of formula (I)

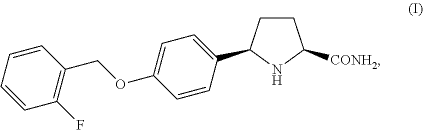 [SMILES: NC(=O)[C@@H]1CC[C@H](c2ccc(OCc3ccccc3F)cc2)N1]

or a pharmaceutically acceptable salt thereof, comprising reacting a compound of formula (XXII) with a compound of formula (VIII), thereby producing a compound of formula (XXIII):

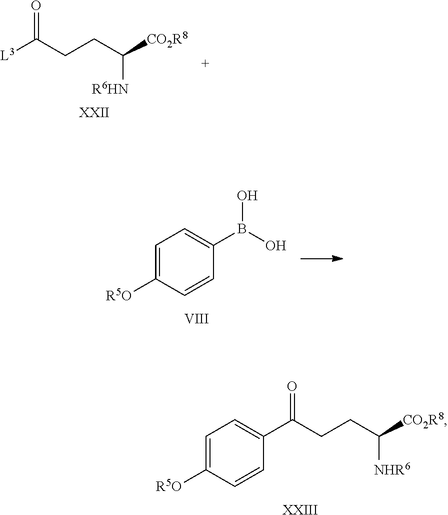

wherein L³ is a leaving group (such as

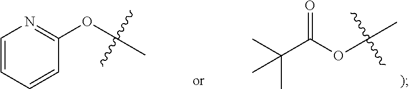

R⁵ is 2-fluorobenzyl or an oxygen-protecting group. In certain preferred embodiments, R⁵ is 2-fluorobenzyl. R⁶ is a resonance-accepting nitrogen-protecting group, such as a nitrogen-protecting group selected from: tert-butyloxycarbonyl (Boc); 9-fluorenylmethyloxycarbonyl (Fmoc); acetyl (Ac); benzoyl (Bz); carbamates; tosyl (Ts); a sulfonamide selected from Nosyl and Nps and trifluoroacetyl. In certain preferred embodiments, R⁶ is tert-butyloxycarbonyl (Boc). In certain embodiments, R⁸ is C₁₋₆ alkyl, C₂₋₆ alkenyl, or C₂₋₆alkynyl, most preferably methyl. In certain embodiments, the process is for preparing a compound of formula (XXIII).

In addition, the present disclosure provides processes for preparing a compound of formula (I)

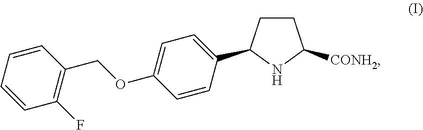 [SMILES: NC(=O)[C@@H]1CC[C@H](c2ccc(OCc3ccccc3F)cc2)N1]

or a pharmaceutically acceptable salt thereof, comprising reacting a compound of formula (VIII) with a compound of formula IX thereby producing a compound of formula X):

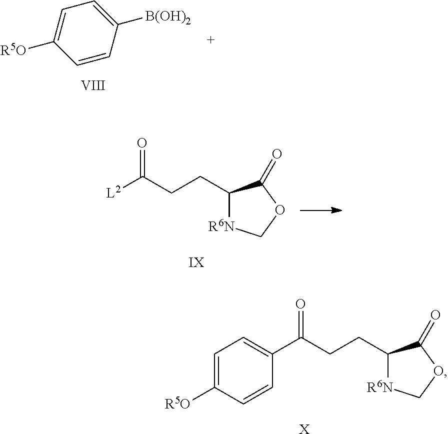

wherein L² is hydroxyl or a leaving group; In certain preferred embodiments, L² is

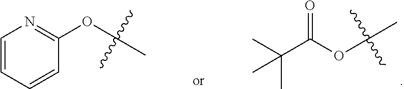

In certain embodiments, R⁵ is 2-fluorobenzyl or an oxygen-protecting group, preferably 2-fluorobenzyl. R⁶ is a resonance-accepting nitrogen-protecting group, such as a nitrogen-protecting group selected from: tert-butyloxycarbonyl (Boc); 9-fluorenylmethyloxycarbonyl (Fmoc); acetyl (Ac); benzoyl (Bz); carbamates; tosyl (Ts); a sulfonamide selected from Nosyl and Nps and trifluoroacetyl. In certain preferred embodiments, R⁴ is tert-butyloxycarbonyl (Boc). In certain embodiments, the process is for preparing a compound of formula (X).

Provided here also includes compounds of formula (XXIV), formula (XXV), formula (XXVI), formula (XXVII)

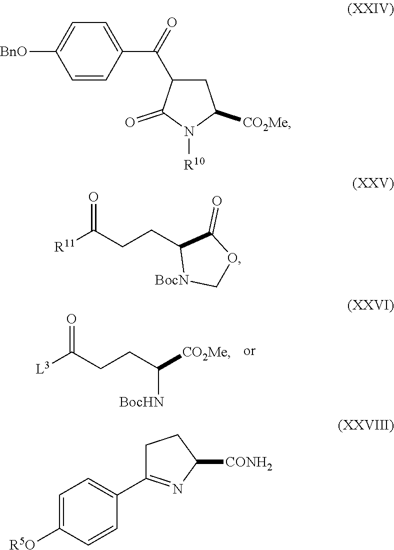

or a pharmaceutically acceptable salt thereof, wherein R¹⁰ is hydrogen or a resonance-accepting nitrogen-protecting group. In certain referred embodiments, R¹⁰ is hydrogen or ten-butyloxycarbonyl (Boc). R¹¹ is

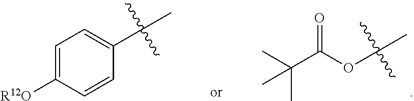

In certain preferred embodiments, R¹² is 2-fluorobenzyl, benzyl or hydroxyl. L³ is a leaving group. In certain preferred embodiments, L³ is

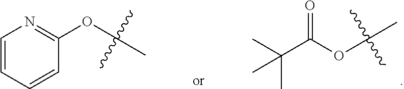

In certain preferred embodiments, R⁵ is 2-fluorobenzyl or an oxygen-protecting group, most preferably 2-fluorobenzyl or benzyl.

### DETAILED DESCRIPTION

In certain aspects, the present disclosure provides processes for preparing a compound of formula (I)

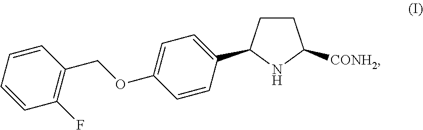 [SMILES: NC(=O)[C@@H]1CC[C@H](c2ccc(OCc3ccccc3F)cc2)N1]

or a pharmaceutically acceptable salt thereof, comprising reacting a compound of formula (II) with a compound of formula (IV) in the presence of a base, thereby producing a compound of formula (V):

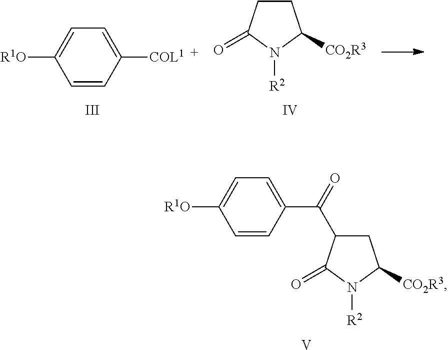

wherein L¹ is a leaving group (such as a halide, e.g., Br or Cl); R¹ is an oxygen-protecting group (such as allyl, benzyl, benzoyl, methoxymethyl, tetrahydropyranyl, tert-butyl, acetyl, silicon-containing protecting group). In certain preferred embodiments, R¹ is benzyl. R² is a resonance-accepting nitrogen-protecting group, such as nitrogen-protecting group selected from: tert-butyloxycarbonyl (Boc); 9-fluorenylmethyloxycarbonyl (Fmoc); acetyl (Ac); benzoyl (Bz); carbamates; tosyl (Ts); a sulfonamide selected from Nosyl and Nps and trifluoroacetyl. In certain preferred embodiments, R² is tert-butyloxycarbonyl (Boc). R³ is C₁₋₆ alkyl, C₂₋₆ alkenyl, or C₂₋₆ alkynyl. In certain preferred embodiments, R³ is methyl. In certain embodiments, the process is for preparing a compound of formula (V).

A “resonance-accepting nitrogen-protecting group”, as the term is used herein, refers to a protecting group that has a n orbital (e.g., an orbital participating in a double or triple bond) capable of accepting electron density from the lone pair of the nitrogen atom to which it is attached, e.g., via a resonance form or tautomer. Carbonyl moieties (e.g., as present in amide, urea, and carbamate functional groups) and sulfonyl moieties (e.g., as present in sulfonamide functional groups) are representative groups capable of accepting electron density from the nitrogen atom in those functional groups.

In certain embodiments, reacting a compound of formula (III) with a compound of formula (IV) in the presence of a base comprising reacting the compound of formula (III) with the compound of formula (IV) in the presence of the base (such as lithium bis(trimethylsilyl) amide) and a solvent (such as tetrahydrofuran).

In certain embodiments, the processes described herein comprise reacting a compound of formula (II) with a carboxyl-activating compound, thereby producing the compound of formula (III):

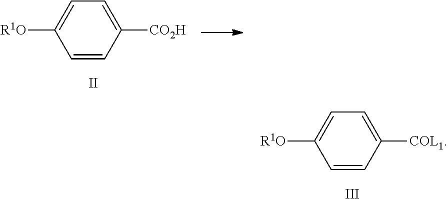

In certain embodiments, reacting a compound of formula (H) with a carboxyl-activating compound comprises reacting the compound of formula (H) with the carboxyl-activating compound in the presence of a solvent. In certain embodiments, the carboxyl-activating compound is oxalyl chloride. In certain embodiments, the solvent is dichloromethane.

A “carboxyl-activating compound”, as the term is used herein, refers to a compound that is capable of reacting with carboxylic acid and providing a leaving group directly attached to the carbonyl. Such leaving groups include but not limited to: chloride, bromide, tosyl, mesyl, trifluoroacetate, etc.

In certain embodiments, the processes described herein comprise deprotecting the compound of formula (V), thereby producing a compound of formula (VI):

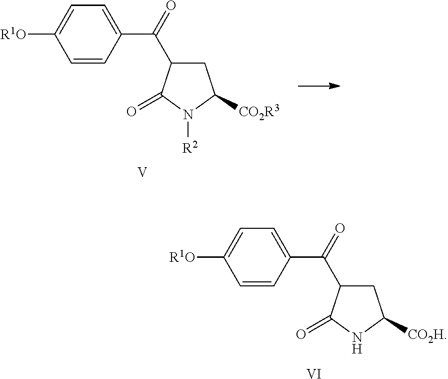

In certain embodiments, deprotecting the compound of formula (V) comprises reacting the compound of formula (V) with an acid. In certain embodiments, deprotecting the compound of formula (V) comprises reacting the compound of formula (V) with an acid in the presence of a solvent. In certain embodiments, the acid is hydrochloric acid. In certain embodiments, the solvent is acetone.

In certain aspects, the present disclosure provides processes for preparing a compound of formula (I)

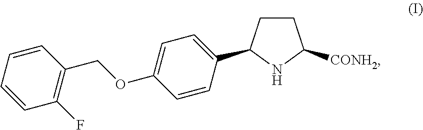 [SMILES: NC(=O)[C@@H]1CC[C@H](c2ccc(OCc3ccccc3F)cc2)N1]

or a pharmaceutically acceptable salt thereof, comprising deprotecting a compound of formula (V), thereby producing a compound of formula (VI):
wherein:

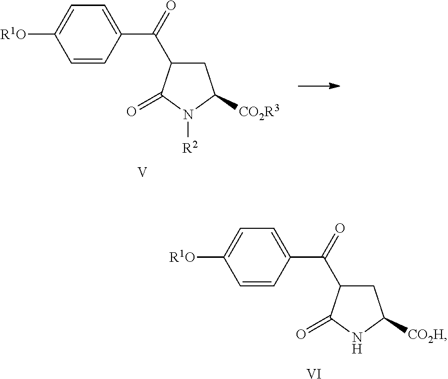

R¹ is an oxygen-protecting group; R² is a resonance-accepting nitrogen-protecting group; and R³ is C₁₋₆ alkyl, C₂₋₆ alkenyl, or C₂₋₆ alkynyl. In certain embodiments, the process is for preparing a compound of formula (VI).

In certain embodiments, the processes described herein comprise reacting the compound of formula (VI) with an acid (such as hydrochloric acid), thereby producing a compound (1), or a salt thereof:

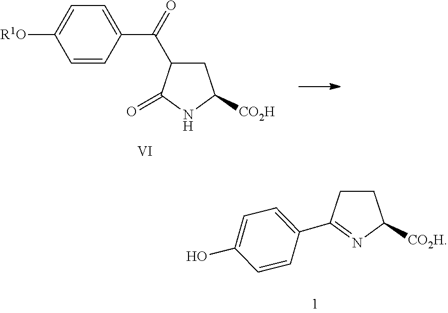

In certain embodiments, the processes described herein comprise reacting the compound (1), or a salt thereof, with methanol, thereby producing a compound (2), or a salt thereof:

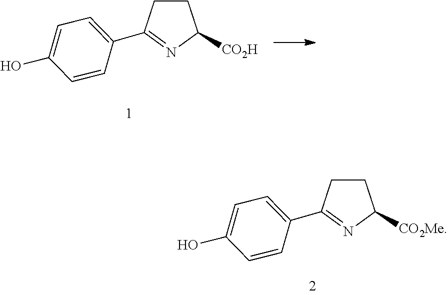 [SMILES: O=C(O)[C@@H]1CCC(c2ccc(O)cc2)=N1 ; COC(=O)[C@@H]1CCC(c2ccc(O)cc2)=N1]

In certain embodiments, reacting the compound (1), or a sak thereof, with methanol comprise reacting the compound (1), or a salt thereof, with methanol in the presence of an acid (such as concentrated hydrochloric acid).

In certain embodiments, the processes described herein comprise reacting the compound (2), or a salt thereof, with hydrogen gas with a compound that provides a resonance-accepting nitrogen-protecting group in the presence of a catalyst, thereby producing a compound of formula (I)

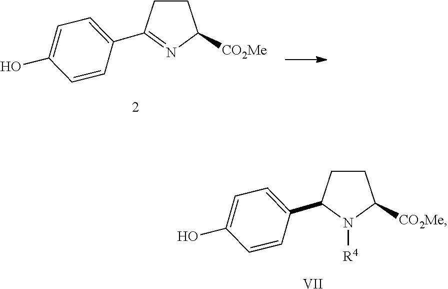

wherein R⁴ is a resonance-accepting nitrogen-protecting group, e.g, a nitrogen-protecting group selected from tert-butyloxycarbonyl (Boc); 9-fluorenylmethyloxycarbonyl (Fmoc); acetyl (Ac); benzoyl (Bz); carbamates; tosyl (Ts); a sulfonamide selected from Nosyl and Nps; and trifluoroacetyl. In certain preferred embodiments, R⁴ is tert-butyloxycarbonyl. In certain embodiments, such as when 2 is provided as an HCl salt, the processes described herein further comprise reacting 2 with an amine base, such as triethylamine.

In certain embodiments, reacting the compound (2) with hydrogen gas with a compound that provides a resonance-accepting nitrogen-protecting group in the presence of a catalyst comprises reacting the compound (2) with hydrogen gas and a compound that provides a resonance-accepting nitrogen-protecting group in the presence of the catalyst (such as Pd(OH)₂/C, e.g., Pd(OH)₂/C) and a solvent (such as methanol).

In certain embodiments, the processes described herein comprise performing two or more of the reactions described above sequentially. In certain such embodiments, the processes described herein comprise:

reacting a compound of formula (II) with a carboxyl-activating compound, thereby producing the compound of formula (III):

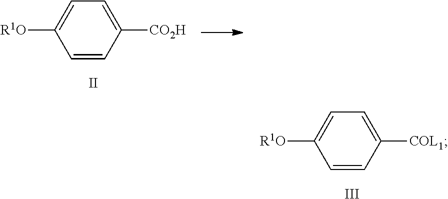

reacting a compound of formula (III) with a compound of formula (IV) in the presence of a base, thereby producing a compound of formula (V):

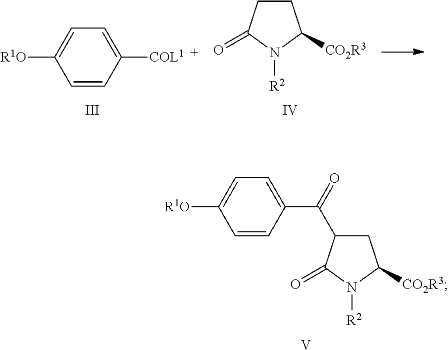

deprotecting the compound of formula (V), thereby producing a compound of formula (VI):

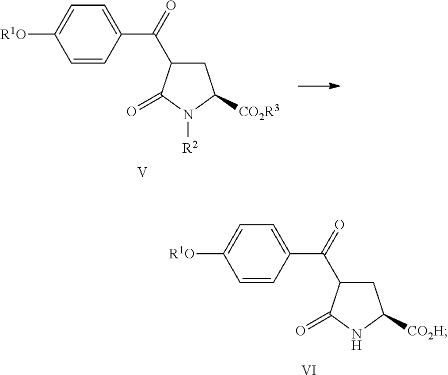

reacting the compound of formula (VI) with an acid, thereby producing a compound (1), or a salt thereof:

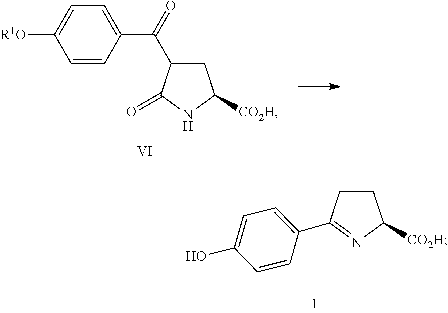

reacting the compound (1), or a salt thereof, with methanol, thereby producing a compound (2), or a salt thereof:

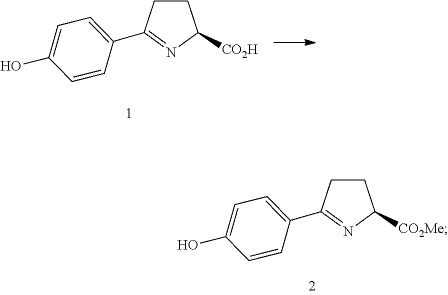 [SMILES: O=C(O)[C@@H]1CCC(c2ccc(O)cc2)=N1 ; COC(=O)[C@@H]1CCC(c2ccc(O)cc2)=N1]

and

reacting the compound (2), or a salt thereof, with hydrogen gas, a compound that provides a resonance-accepting nitrogen-protecting group in the presence of a catalyst, thereby producing a compound of formula (VII):

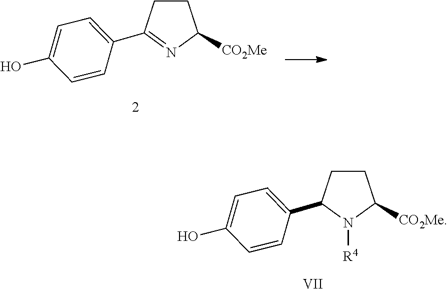

In certain aspects, the present disclosure provides processes for preparing a compound of formula (I)

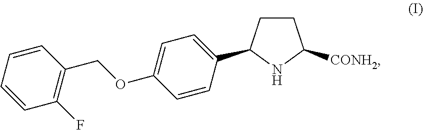 [SMILES: NC(=O)[C@@H]1CC[C@H](c2ccc(OCc3ccccc3F)cc2)N1]

or a pharmaceutically acceptable salt thereof, comprising reacting a compound of formula (VII) with a compound of formula (IX), thereby producing a compound of formula (X):

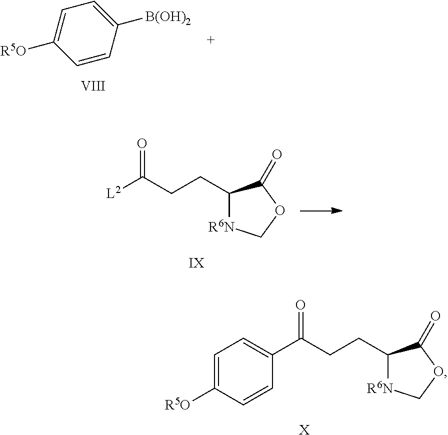

wherein L² is hydroxyl or a leaving group; In certain preferred embodiments, L² is

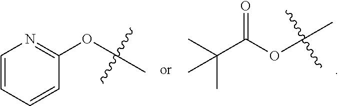

In certain embodiments, R⁵ is 2-fluorobenzyl or an oxygen-protecting group, preferably 2-fluorobenzyl. R⁶ is a resonance-accepting nitrogen-protecting group, such as a nitrogen-protecting group selected from: tert-butyloxycarbonyl (Boc); 9-fluorenylmethyloxycarbonyl (Fmoc); acetyl (Ac); benzoyl (Bz); carbamates; tosyl (Ts); a sulfonamide selected from Nosyl and Nps and trifluoroacetyl. In certain preferred embodiments, R⁶ is tert-butyloxycarbonyl (Boc). In certain embodiments, the process is for preparing a compound of formula (X).

In certain embodiments, reacting a compound of formula (VIII) with a compound of formula (IX) comprises reacting a compound of formula (VIII) with a compound of formula (IX) in the presence of a palladium coupling reagent. In certain embodiments, reacting a compound of formula (VIII) with a compound of formula (IX) comprises reacting a compound of formula (VIII) with a compound of formula (IX) in the presence of a palladium coupling reagent (such as Pd(OAc)₂ and PPh₃) and a solvent (such as tetrahydrofuran). In certain embodiments, the process further comprises preparing the palladium coupling reagent by reacting Pd(OAc)₂ with PPh₃.

In certain embodiments, the processes described herein comprise reacting the compound of formula (X) with an acid, thereby producing a compound of formula (XI):

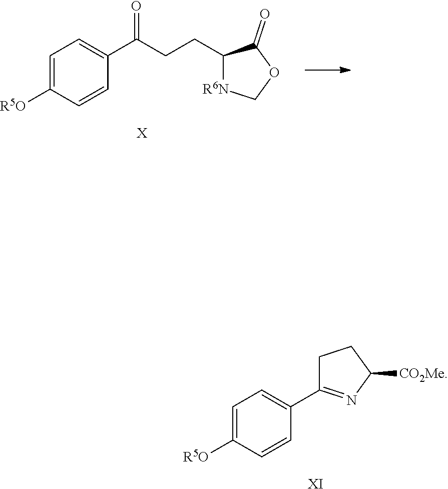

In certain embodiments, reacting the compound of formula (X) with an acid comprises reacting the compound of formula (X) with an acid (such as methansulfonic acid and/or sulfuric acid) in the presence of a solvent. In certain embodiments, reacting the compound of formula (X) with an acid comprises reacting the compound of formula (X) with an acid (such as methansulfonic acid and/or sulfuric acid) in the presence of methanol, optionally in the presence of other solvents.

In certain aspects, the current disclosure provides processes for preparing a compound of formula (I)

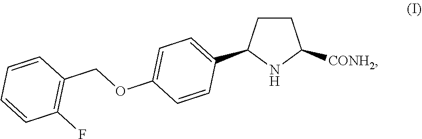 [SMILES: NC(=O)[C@@H]1CC[C@H](c2ccc(OCc3ccccc3F)cc2)N1]

or a pharmaceutically acceptable salt thereof, comprising reacting a compound of formula (X) with an acid, such as methanesulfonic acid and/or sulfuric acid, thereby producing a compound of formula (XI):

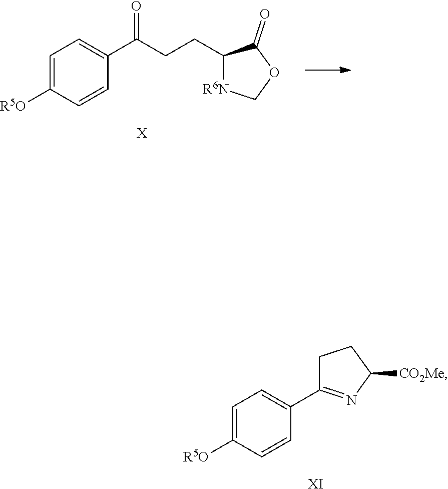

wherein: R⁵ is 2-fluorobenzyl or an oxygen-protecting group; and R⁶ is a resonance-accepting nitrogen-protecting group. In certain embodiments; the process is for preparing a compound of formula (XI)

In certain embodiments, the processes described herein comprise reacting the compound of formula (XI) with NH₄OH, thereby producing a compound of formula (XII):

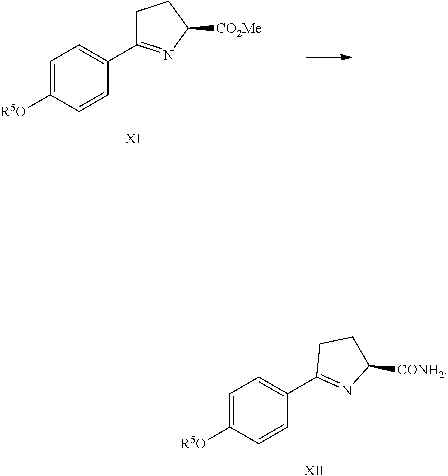

In certain embodiments, reacting the compound of formula (XI) with NH₄OH comprises reacting the compound of formula (XI) with NH₄OH in the presence of a solvent (such as tetrahydrofuran).

In certain aspects, the current disclosure provides processes for preparing a compound of formula (I)

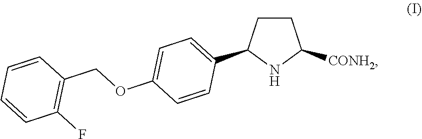 [SMILES: NC(=O)[C@@H]1CC[C@H](c2ccc(OCc3ccccc3F)cc2)N1]

or a pharmaceutically acceptable salt thereof, comprising reacting a compound of formula (XI) with NH₄OH, thereby producing a compound of formula (XII):

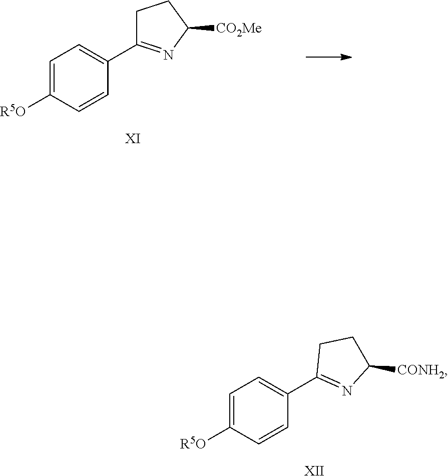

wherein R⁵ is 2-fluorobenzyl or an oxygen-protecting group. In certain embodiments; the process is for preparing a compound of formula (XII)

In certain aspects, the present disclosure provides processes for preparing a compound of formula (I)

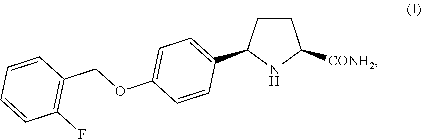 [SMILES: NC(=O)[C@@H]1CC[C@H](c2ccc(OCc3ccccc3F)cc2)N1]

or a pharmaceutically acceptable salt thereof, comprising reacting a compound of formula (XXII) with a compound of formula (VIII) thereby producing a compound of formula XXIII):

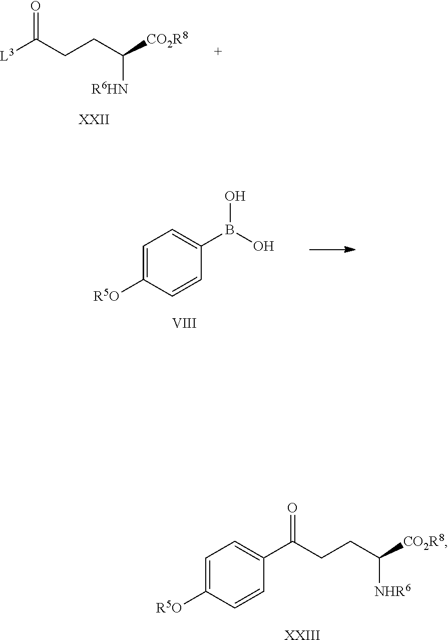

wherein L³ is a leaving group (such as

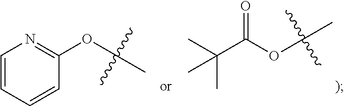

R⁵ is 2-fluorobenzyl or an oxygen-protecting group. In certain preferred embodiments, R⁵ is 2-fluorobenzyl. In certain preferred embodiments, R⁶ is a resonance-accepting nitrogen-protecting group, such as a nitrogen-protecting group selected from: tert-butyloxycarbonyl (Boc); 9-fluorenylmethyloxycarbonyl (Fmoc); acetyl (Ac); benzoyl (Bz); carbamates; tosyl (Ts); a sulfonamide selected from Nosyl and Nps and trifluoroacetyl. In certain preferred embodiments, R⁶ is tert-butyloxycarbonyl (Boc). In certain embodiments, R⁸ is C₁₋₆ alkyl, C₂₋₆alkenyl, or C₂₋₆ alkynyl, most preferably methyl. In certain embodiments, the process is for preparing a compound of formula (XXI).

In certain embodiments, reacting a compound of formula (XXII) with a compound of formula (VIII) comprises reacting the compound of formula (XXII) with the compound of formula (VIII) in the presence of a palladium coupling reagent. In certain embodiments, reacting a compound of formula (XXII) with a compound of formula (VIII) comprises reacting the compound of formula (XXII) with the compound of formula (VIII) in the presence of a palladium coupling reagent (such as Pd(OAc)₂ and PPh₃) and a solvent (such as 1,4-dioxane). In certain embodiments, the process further comprises preparing the palladium coupling reagent by reacting Pd(OAc)₂ with PPh₃

In certain embodiments, the processes described herein comprise reacting the compound of formula (XXIII) with NH₄OH, thereby producing a compound of formula (XIII):

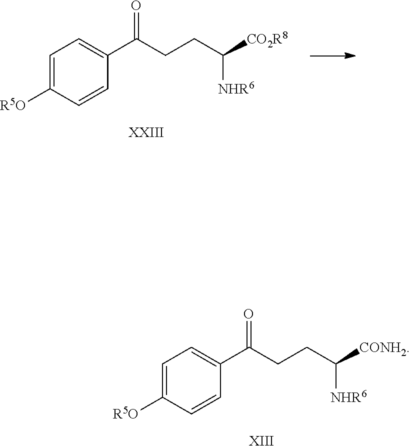

In certain embodiments, reacting the compound of formula (XXI) with NH₄OH comprises reacting the compound of formula (X) with NH₄OH in the presence of a solvent (such as tetrahydrofuran/methanol).

In certain aspects, the current disclosure provides processes for preparing a compound of formula (I)

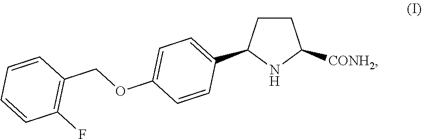 [SMILES: NC(=O)[C@@H]1CC[C@H](c2ccc(OCc3ccccc3F)cc2)N1]

or a pharmaceutically acceptable salt thereof, comprising reacting a compound of formula (XXIII) with NH₄OH, thereby producing a compound of formula (XIII):

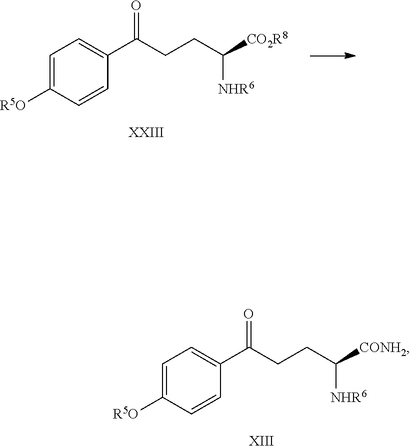

wherein R⁵ is 2-fluorobenzyl or an oxygen-protecting group; R⁶ is a resonance-accepting nitrogen-protecting group; and R⁸ is C₁₋₆ alkyl, C₂₋₆alkenyl, or C₂₋₆ alkynyl. In certain embodiments, the process is for preparing a compound formula (XIII)

In certain embodiments, the processes described herein comprise reacting the compound of formula (X) with NH₄OH, thereby producing a compound of formula (XIII):

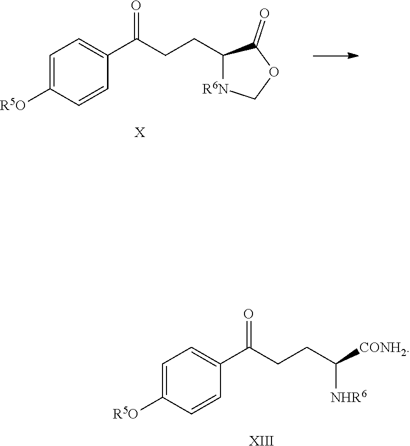

In certain embodiments, reacting the compound of formula (X) with NH₄OH comprises reacting the compound of formula (X) with NH₄OH in the presence of a solvent (such as tetrahydrofuran).

In certain embodiments, the processes described herein comprise deprotecting the compound of formula (XIII), thereby producing a compound of formula (XII):

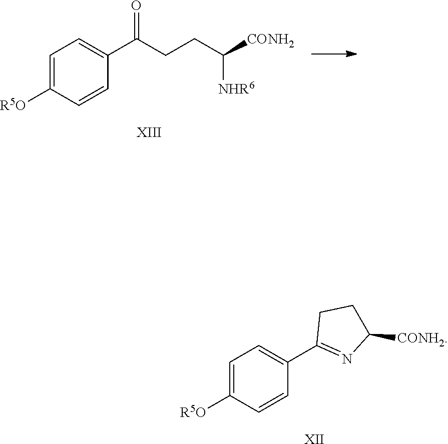

In certain embodiments, deprotecting the compound of formula (XIII) comprises reacting the compound of formula (XIII) with an acid. In certain embodiments, deprotecting the compound of formula (XIII) comprises reacting the compound of formula (XIII) with an acid (such as hydrochloric acid) in the presence of a solvent (such as tetrahydrofuran).

In certain aspect, the current disclosure provides processes for preparing a compound of formula (I)

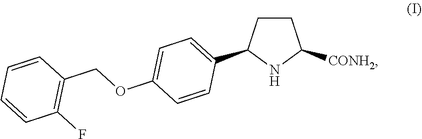 [SMILES: NC(=O)[C@@H]1CC[C@H](c2ccc(OCc3ccccc3F)cc2)N1]

or a pharmaceutically acceptable salt thereof, comprising deprotecting a compound of formula (XIII), thereby producing a compound of formula (XII):

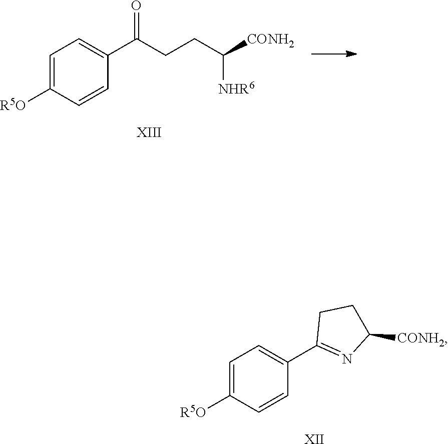

wherein R⁵ is 2-fluorobenzyl or an oxygen-protecting group; and R⁶ is a resonance-accepting nitrogen-protecting group. In certain embodiments; the process is for preparing a compound of formula (XII).

In certain embodiments, the processes described herein comprise reacting the compound of formula (XII) with hydrogen gas in the presence of a catalyst, thereby producing a compound of formula (XIV):

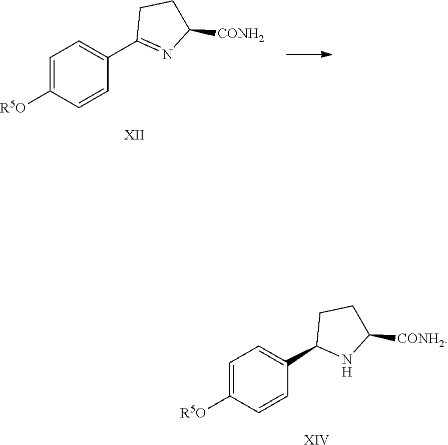

In certain embodiments, reacting the compound of formula (XII) with hydrogen gas in the presence of a catalyst comprises reacting the compound of formula (XII) with hydrogen gas in the presence of a catalyst (such as PtO₂) and a solvent (such as methanol).

In certain aspects, the current disclosure provides processes for preparing a compound of formula (I)

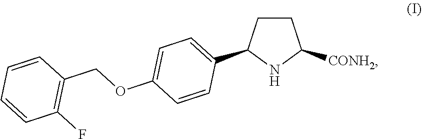 [SMILES: NC(=O)[C@@H]1CC[C@H](c2ccc(OCc3ccccc3F)cc2)N1]

or a pharmaceutically acceptable salt thereof, comprising reacting a compound of formula (Xli) with hydrogen gas in the presence of a catalyst, such as PtO₂, thereby producing a compound of formula (XIV):

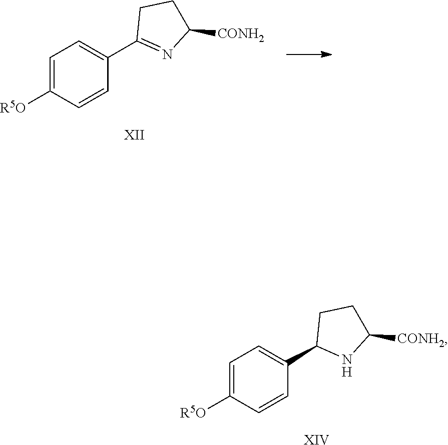

wherein R⁵ is 2-fluorobenzyl or an oxygen-protecting group. In certain embodiments; the process is for preparing a compound of formula (XIV)

In certain embodiments, the processes described herein comprise reacting the compound of formula (XIV) with hydrochloric acid, thereby producing a compound of formula (XV):

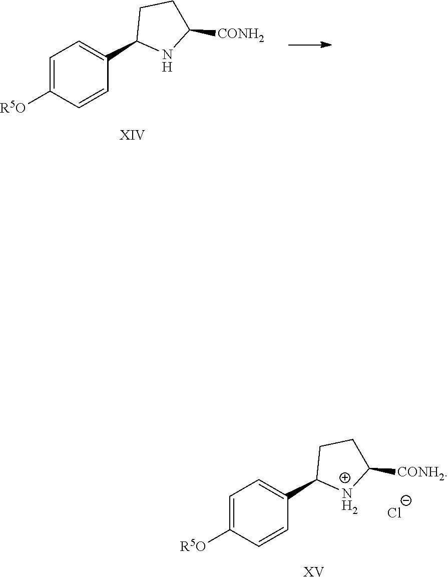

In certain embodiments, reacting the compound of formula (XIV) with hydrochloric acid comprises reacting the compound of formula (XIV) with hydrochloric acid in the presence of a solvent (such as methanol). In certain embodiments, XI is reacted to form XIV in the presence of HCl such that XIV forms XV quickly after being formed.

In certain embodiments, R⁵ is 2-fluorobenzyl or an oxygen-protecting group selected from: benzyl, benzoyl, methoxymethyl, tetrahydropyranyl, tert-butyl, acetyl, and silicon-containing protecting group. In certain preferred embodiments, R⁵ is 2-fluorobenzyl.

In certain embodiments, the processes described herein comprise reacting the compound of formula (XII) with hydrogen gas in the presence of a catalyst, thereby producing a compound of formula (XVI):

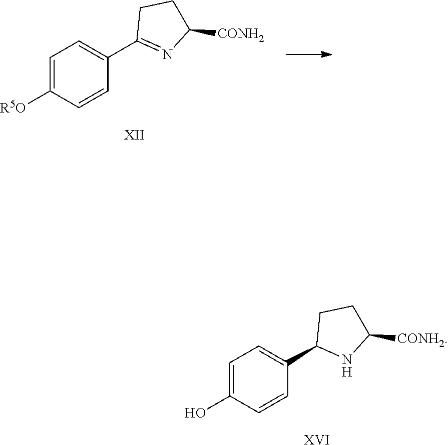

In certain embodiments, reacting the compound of formula (XII) with hydrogen gas in the presence of a catalyst comprises reacting the compound of formula (XII) with hydrogen gas in the presence of a catalyst (such as Pd(OH)₂/C, e.g., 20% wt % Pd(OH)₂/C) and a solvent (such as tetrahydrofuran and/or methanol).

In certain embodiments, R⁵ is 2-fluorobenzyl or an oxygen-protecting group. In certain embodiments, R⁵ is an oxygen-protecting group selected from: benzyl, benzoyl, methoxymethyl, tetrahydropyranyl, tert-butyl, acetyl, and silicon-containing protecting group. In certain preferred embodiments, R⁵ is benzyl.

In certain aspects, the current disclosure provides processes for preparing a compound of formula (I)

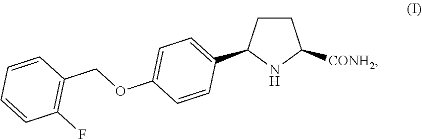 [SMILES: NC(=O)[C@@H]1CC[C@H](c2ccc(OCc3ccccc3F)cc2)N1]

or a pharmaceutically acceptable salt thereof, comprising reacting a compound of formula (XII) with hydrogen gas in the presence of a catalyst, thereby producing a compound of formula (XVI):

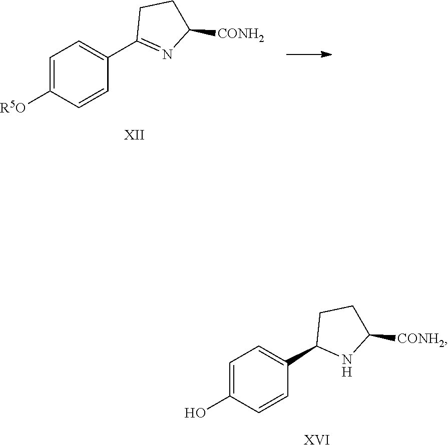

wherein R⁵ is 2-fluorobenzyl or an oxygen-protecting group. In certain embodiments, the process is for preparing a compound of formula (XVI).

In certain embodiments, the processes described herein comprise reacting the compound of formula (XVI) with a compound that provides a resonance-accepting nitrogen-protecting group, thereby producing a compound of formula (XVII):

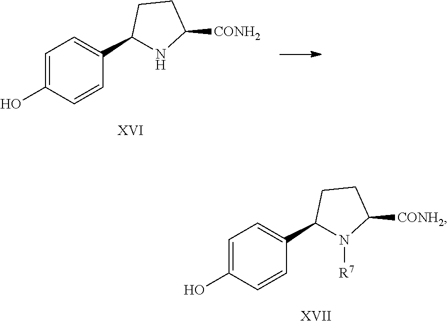

wherein R⁷ is a resonance-accepting nitrogen-protecting group, e.g, a nitrogen-protecting group selected from: tert-butyloxycarbonyl (Boc); 9-fluorenylmethyloxycarbonyl (Fmoc); acetyl (Ac); benzoyl (Bz); carbamates; tosyl (Ts); a sulfonamide selected from Nosyl and Nps and trifluoroacetyl. In certain preferred embodiments, R⁷ is tert-butyloxycarbonyl (Boc).

In certain embodiments, reacting the compound of formula (XVI) with a compound that provides a resonance-accepting nitrogen-protecting group comprises reacting the compound of formula (XVI) with the compound that provides a resonance-accepting nitrogen-protecting group (such as di-tert-butyldicarbonate) in the presence of a solvent (such as tetrahydrofuran and/or methanol).

In certain embodiments, XII is reacted to form XVI in the presence of the compound that provides a resonance-accepting nitrogen-protecting group such that XVI reacts to form XVII quickly after being formed.

In certain embodiments, the processes described herein comprise reacting the compound of formula (XVII) with a compound of formula (XVIII), thereby producing a compound of formula (XIX):

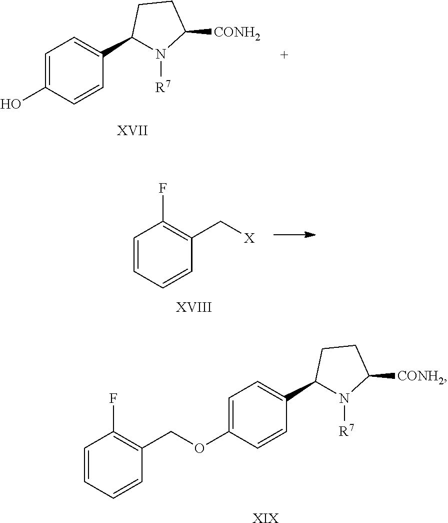

wherein X is a halogen. In certain preferred embodiments, X is a bromide.

In certain embodiments, reacting the compound of formula (XVII) with a compound of formula (XVIII) comprises reacting the compound of formula (XVII) with the compound of formula (XVIII) in the presence of a base. In certain embodiments, reacting the compound of formula (XVII) with a compound of formula (XVIII) comprises reacting the compound of formula (XVII) with the compound of formula (XVIII) in the presence of a base (such as sodium methoxide) and a solvent (such as formamide and/or dimethylformamide).

In certain embodiments, the processes described herein comprise deprotecting the compound of formula (XIX), thereby producing a compound of formula (XX):

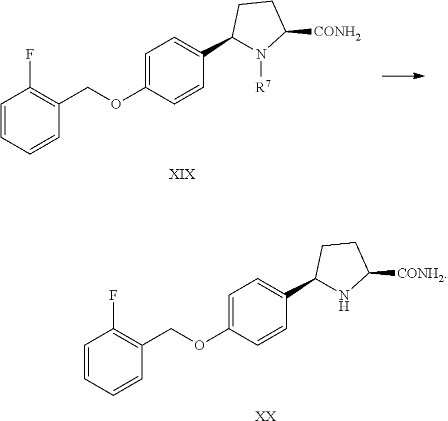

In certain embodiments, deprotecting the compound of formula (XIX) comprises deprotecting the compound of formula (XIX) in the presence of an acid. In certain embodiments, deprotecting the compound of formula (XIX) comprises deprotecting the compound of formula (XIX) in the presence of an acid (such as hydrochloric acid, methansulfonic acid, or sulfuric acid) and a solvent (such as tetrahydrofuran, acetonitrile, and/or methanol).

In certain embodiments, the processes described herein comprise reacting the compound of formula (XX) with hydrochloric acid, thereby producing a compound of formula (XXI):

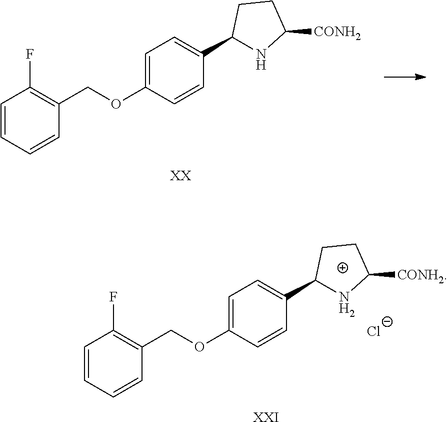

In certain embodiments, reacting the compound of formula (XX) with hydrochloric acid comprises reacting the compound of formula (XX) with hydrochloric acid in the presence of a solvent (such as tetrahydrofuran, isopropanol, and/or methanol).

In certain embodiments, the processes described herein comprise performing two or more of the reactions described above sequentially. In certain such embodiments, the processes described herein comprise:

reacting a compound of formula (VIII) with a compound of formula (IX), thereby producing a compound of formula (X):

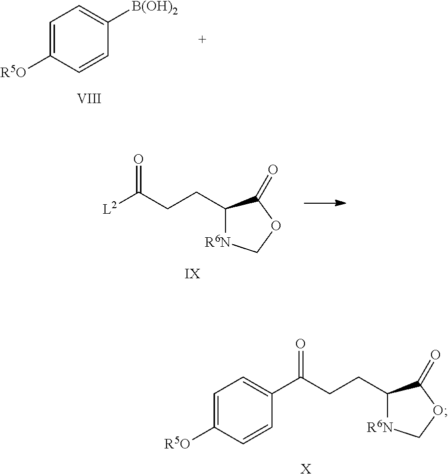

reacting the compound of formula (X) with an acid, thereby producing a compound of formula (XI):

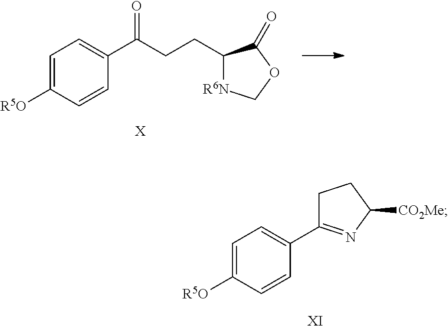

reacting the compound of formula (XI) with NH₄OH, thereby producing a compound of formula (XII):

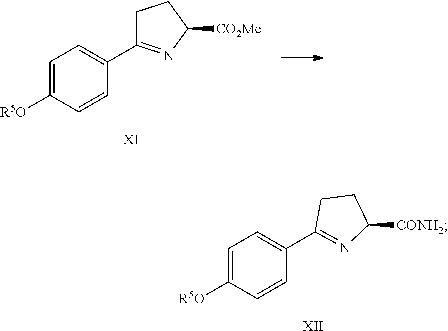

reacting the compound of formula (XII) with hydrogen gas in the presence of a catalyst, thereby producing a compound of formula (XIV):

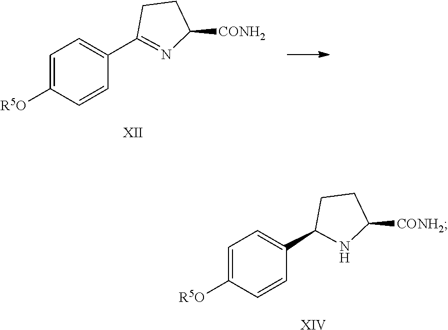

reacting the compound of formula (XIV) with hydrochloric acid, thereby producing a compound of formula (XV):

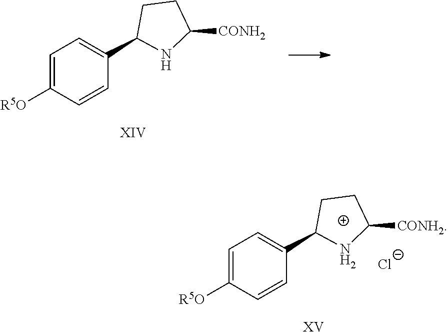

In certain embodiments, the processes described herein comprise performing two or more of the reactions described above sequentially. In certain such embodiments, the processes described herein comprise:

reacting a compound of formula (XXII) with a compound of formula (VIM), thereby producing a compound of formula (XXIII):

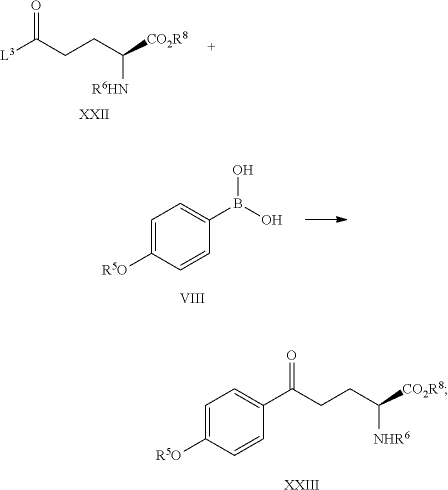

reacting the compound of formula (XXI) with NH₄OH, thereby producing a compound of formula (XIII):

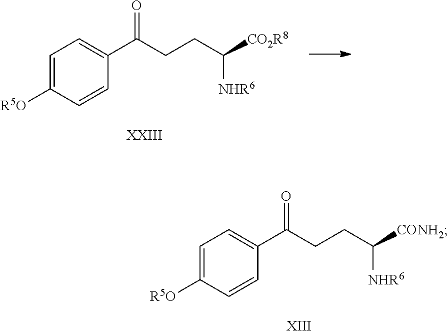

deprotecting the compound of formula (XIII), thereby producing a compound of formula (XII):

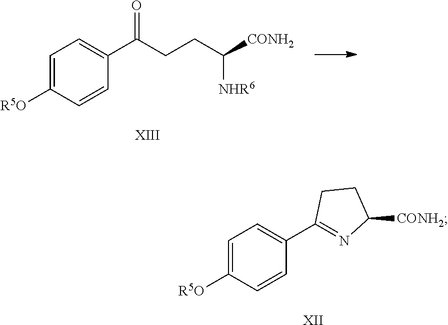

reacting the compound of formula (XII) with hydrogen gas in the presence of a catalyst, thereby producing a compound of formula (XVI):

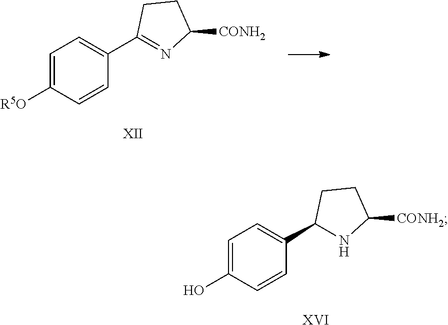

reacting the compound of formula (XVI) with a compound that provides a resonance-accepting nitrogen-protecting group, thereby producing a compound of formula (XVII):

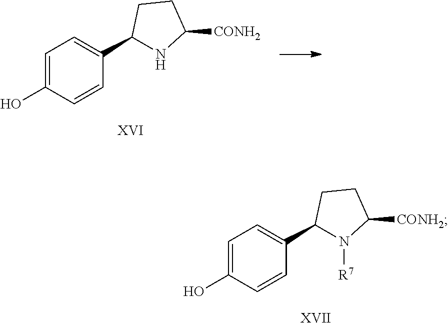

reacting the compound of formula (XVII) with a compound of formula (XVIII), thereby producing a compound of formula (XIX):

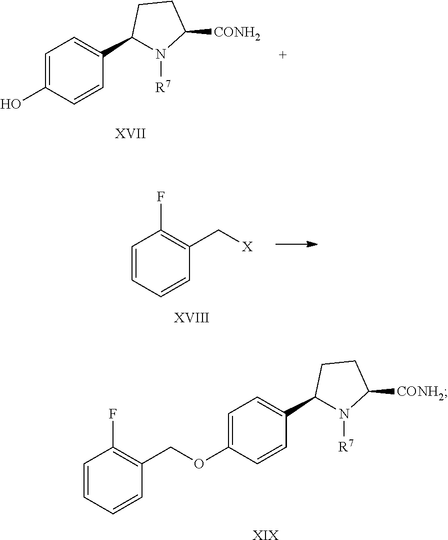

deprotecting the compound of formula (XIX), thereby producing a compound of formula (XX):

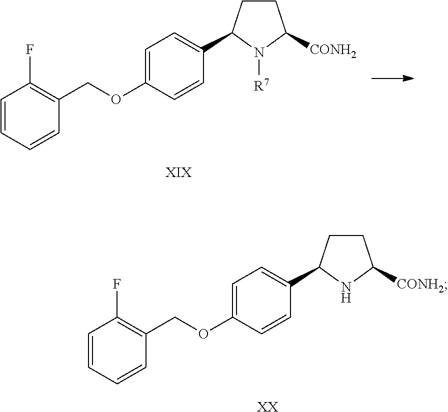

and

reacting the compound of formula (XX) with hydrochloric acid, thereby producing a compound of formula (XXI):

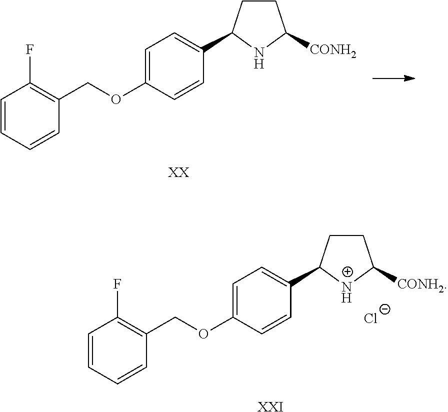

In certain aspects, the present disclosure provides compounds of formula (XXIV), formula (XXV), formula (XXVI), formula (XXVII), or formula (XXVIII)

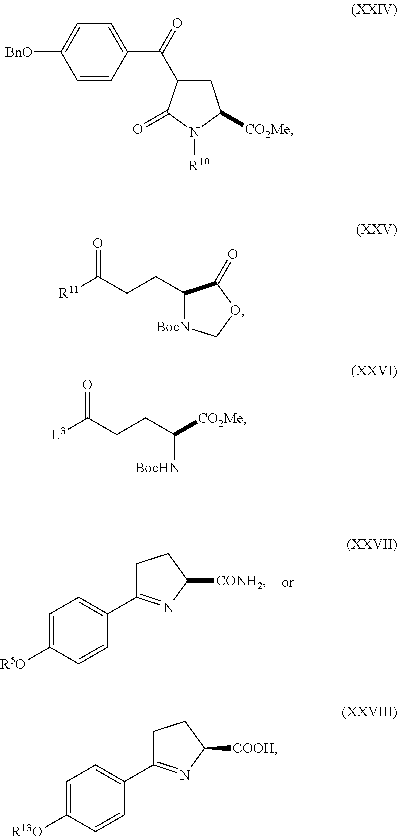

or a pharmaceutically acceptable salt thereof, wherein R¹⁰ is hydrogen or a resonance-accepting nitrogen-protecting group. In certain preferred embodiments, R¹⁰ is hydrogen or tert-butyloxycarbonyl (Boc). R¹¹ is

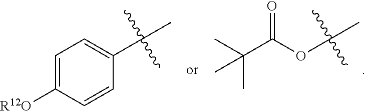

In certain preferred embodiments, R¹² is 2-fluorobenzyl, benzyl, or hydroxyl. L³ is a leaving group. In certain preferred embodiments, L³ is

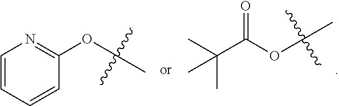

R⁵ is 2-fluorobenzyl, or an oxygen-protecting group. In certain preferred embodiments, R⁵ is 2-fluorobenzyl or benzyl. R³ is hydrogen, benzyl, 2-fluorobenzyl, or an oxygen-protecting group. In certain preferred embodiments, R¹³ is hydrogen.

In certain embodiments, the compound has the structures:

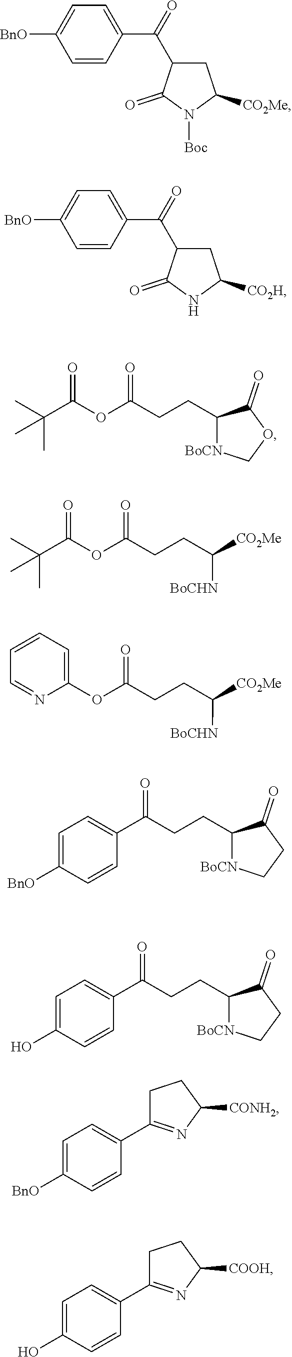

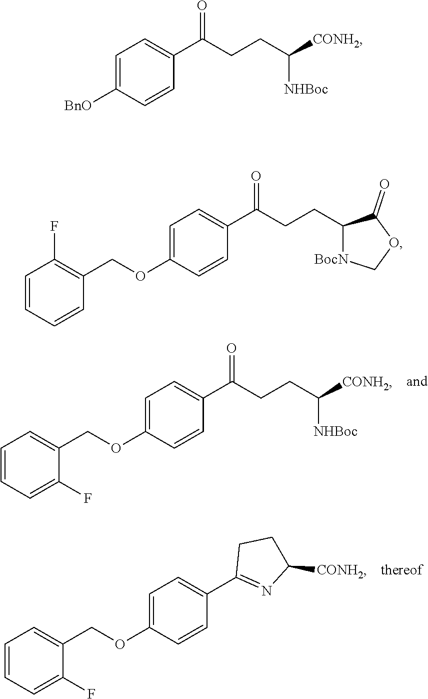 [SMILES: CC(C)(C)OC(=O)N[C@@H](CCC(=O)c1ccc(OCc2ccccc2)cc1)C(N)=O ; CC(C)(C)OC(=O)N1COC(=O)[C@@H]1CCC(=O)c1ccc(OCc2ccccc2F)cc1 ; CC(C)(C)OC(=O)N[C@@H](CCC(=O)c1ccc(OCc2ccccc2F)cc1)C(N)=O ; NC(=O)[C@@H]1CCC(c2ccc(OCc3ccccc3F)cc2)=N1]

or a salt

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, Calif. (1985).

All of the above, and any other publications, patents and published patent applications referred to in this application are specifically incorporated by reference herein. In case of conflict, the present specification, including its specific definitions, will control.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

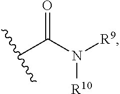

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

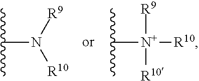

wherein R⁹, R¹⁰, and R¹⁰, each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

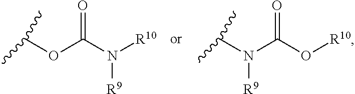

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic.

Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group. The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

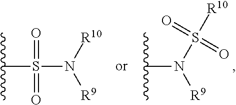

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹

wherein R⁹ represents a hydrocarbyl.

The term “thioether”, as used herein, is equivalent to an ether, wherein the oxygen is replaced with a sulfur.

The term “urea” is art-recognized and may be represented by the general formula

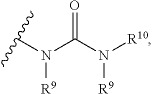

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Salt” is used herein to refer to an acid addition salt or a basic addition salt.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g, WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### Exemplary Materials and Methods

Unless otherwise stated, reactions were performed in glassware fitted with rubber septa under nitrogen atmosphere and were stirred with Teflon-coated magnetic stirring bars. All solvents and reagents were used as received from commercial sources, unless otherwise noted.

Reaction temperatures above 23° C. refer to water bath temperatures. Thin layer chromatography (TLC) was performed using SiliCycle silica gel 60 F-254 precoated plates (0.25 mm) and visualized under UV irradiation, with a cerium ammonium molybdate (CAM) stain or a potassium permanganate (KMnO₄) stain. SiliCycle Silica-P silica gel (particle size 40-63 μm) was used for flash column chromatography. ¹H and ¹³C NMR spectra were recorded using Bruker AV-500, DRX-500, and AV-400 MHz spectrometers, with ¹³C NMR spectroscopic operating frequencies of 125, 125, and 100 MHz, respectively. Chemical shifts (δ) are reported in parts per million (ppm) relative to the residual protonated solvent: CDCl₃ signal (δ=7.26 for ¹H NMR; δ=77.2 for ¹³C NMR), C₆D₆ signal (δ=7.16 for ¹H NMR; δ=128.1 for ¹³C NMR), DMSO-d₆ (δ=2.50 for ¹H NMR; 5=39.5 for ¹³C NMR). Data for ¹H NMR spectra are reported as follows: chemical shift, multiplicity, coupling constants (Hz), and number of hydrogen atoms.

Data for ¹³C NMR spectra are reported in terms of chemical shift. The following abbreviations are used to describe the multiplicities: s=singlet; d=doublet; t=triplet; q=quartet; quint=quintet; m=multiplet; br=broad. HRMS (ESI) was performed using a Waters LCT Premier spectrometer equipped with ACQUITY UPLC system and autosampler. HRMS (DART) was performed using a Thermo Fisher Scientific Exactive Plus spectrometer equipped with an IonSense ID-CUBE DART source. X-ray crystallographic data were collected using a Bruker SMART CCD-based diffractometer equipped with a low-temperature apparatus operated at 100 K. Abbreviations: Ac, acetyl; Bn, benzyl; BOC, tert-butoxycarbonyl; Bu, butyl; DCM, dichloromethane; DMSO, dimethyl sulfoxide; Et, ethyl; EtOAc, ethyl acetate; Et₂O, diethyl ether; HAD, hydrogen atom donor; IPA, isopropyl alcohol; LiHMDS, Me, methyl; MeOH, methanol; Ph, phenyl; PhSH, benzenethiol; TBS, tert-butyldimethylsilyl; TEA, trimethylamine; TEMPO, (2,2,6,6-tetramethylpiperidin-1-yl)oxyl; THF, tetrahydrofuran; p-TSA, para-toluenesulfonic acid; Pr, propyl.

### Example 1: Preparation of (2S, 4R/S)-4-(4-(benzyloxy)benzoyl)-5-oxopyrrolidine-2-carboxylic acid

Part A: In Situ Preparation of 4-(benzyloxy)benzoyl chloride

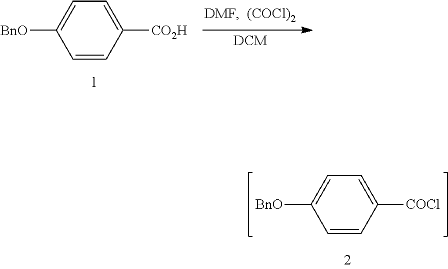

A reactor was charged with 4-(benzyloxy)benzoic acid (161.4 g) and DCM (10V), and N,N-dimethylformamide (0.1 eq) was added. Oxalyl chloride (1.4 eq) was slowly added over about 30 min and the reaction was stirred at ambient temperature for about 100 min. The solution was concentrated to ˜50% of its original volume and fresh DCM (10V) was charged.

The solution was concentrated to ˜33% of its original volume and fresh DCM was charged to bring the preparation to its original volume, and the solution was cooled to 0-5° C. under an inert atmosphere of nitrogen. This DCM solution of the title product was used immediately in the following procedure.

Part B: In Situ Preparation of 1-(tert-butyl) 2-methyl (2S, 4R/S)-4-(4-(benzyloxy)benzoyl)-5-oxopyrrolidine-1,2-dicarboxylate

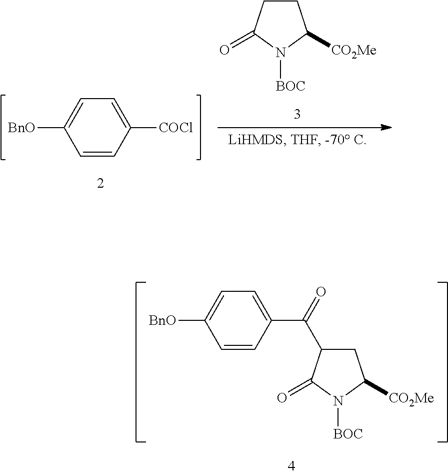

A reactor was charged with 1-(tert-butyl) 2-methyl (S)-5-oxopyrrolidine 1,2-dicarboxylate (132 g) and anhydrous THF (13V) and the solution was cooled to −70° C. under an inert atmosphere of nitrogen. A solution of LiHMDS-THF (1M, 1.1 eq) was added slowly over 35-40 min, and the resulting solution was aged at −70° C. for about 1 h. This solution was cannulated into the 4-(benzyloxy)benzoyl chloride-DCM solution over 25-30 min. The progress of the reaction at 0-5° C. was monitored for completion by HPLC (about 100 min). 20% brine solution (10V) and EtOAc (10V) were added, and the mixture was stirred at ambient temperature for about 10 min. The layers were separated, and the layer was back-extracted with EtOAc (4.5V). The combined organic layers were concentrated in vacuo by rotary evaporation at about 40° C. to near dryness. Acetone (6.8V) was charged and the mixture was agitated on the rotary evaporator, without vacuum, until a uniform slurry was observed. The suspension was concentrated in vacuo by rotary evaporation at about 40° C. to near dryness, and acetone (5.1V) was added to the residue. The mixture was agitated on the rotary evaporator at about 33° C., without vacuum, until a uniform slurry was observed. The solids were removed by filtration, washed with fresh acetone (1.3V) and discarded. The acetone filtrate containing the title product was used immediately in the following procedure.

This preparation was repeated (124 g of 1-(tert-butyl) 2-methyl (S)-5-oxopyrrolidine 1,2-dicarboxylate).

Part C: Preparation of (2S, 4R/S)-4-(4-(benzyloxy)benzoyl)-5-oxopyrrolidine-2-carboxylic acid

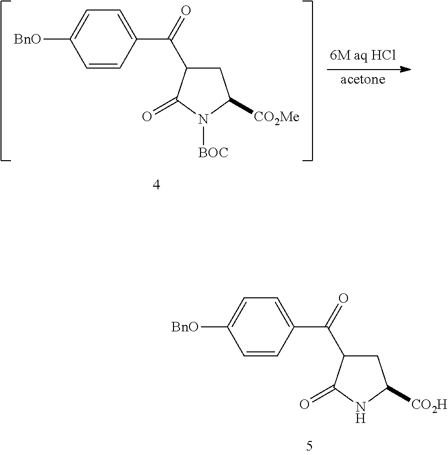

Acetone filtrates from duplicate runs of Part B were charged to a reactor, and the glassware was rinsed forward into the reactor with acetone (0.2V based on 477 g (theory) of the ester). 6M aq HCl (6.8V) was charged and the reaction was heated at 50-55° C. for about 1 h; the progress of the conversion was monitored by HPLC. The mixture was cooled to ambient temperature over about 2.5 h and aged for about 18 h. Solids were isolated by filtration, washed twice with 1:1 (v/v) acetone—water (1.9V) and dried to constant weight under a flow of nitrogen to afford 233 g (59%) of the title compound as a white solid.

### Example 2: Preparation of (S)-5-(4-hydroxyphenyl)-3,4-dihydro-2H-pyrrole-2-carboxylic acid hydrochloride

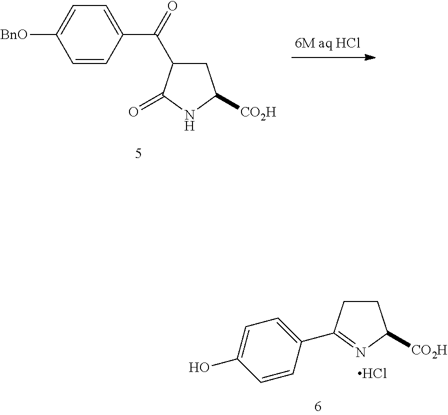 [SMILES: O=C1N[C@H](C(=O)O)CC1C(=O)c1ccc(OCc2ccccc2)cc1 ; O=C(O)[C@@H]1CCC(c2ccc(O)cc2)=N1 ; Cl]

(2S, 4R/S)-4-(4-(benzyloxy)benzoyl)-5-oxopyrrolidine-2-carboxylic acid (116.1 g) was suspended in 6M HCl (20V) and the mixture was heated under reflux for about 2 h with distillate collection in a Dean-Stark trap. The progress of the reaction was monitored for completion by HPLC. The solution was cooled to 50° C., polish filtered and concentrated in vacuo at 55-60° C. to a thick slurry (to a net weight of 1.5× the mass of the input acid). Acetone (10V) was charged, and the suspension was concentrated in vacuo to a net weight of 2× the mass of the input acid. Acetone (20V) was charged and the suspension was stirred at ambient temperature for about 18 h. Solids were isolated by filtration, washed twice with acetone (2V) and dried to constant weight under a flow of nitrogen to afford 71 g (94%) of the title compound as a white solid.

A second run at 116.5 g scale afforded 63.6 g (85%) of the title compound as a white solid.

### Example 3: Preparation of methyl (S)-5-(4-hydroxyphenyl)-3,4-dihydro-2H-pyrrole-2-carboxylate hydrochloride

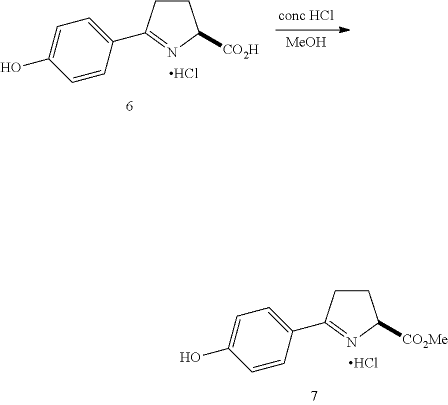 [SMILES: "MeOH" ; O=C(O)[C@@H]1CCC(c2ccc(O)cc2)=N1 ; Cl ; COC(=O)[C@@H]1CCC(c2ccc(O)cc2)=N1 ; Cl]

A reactor was charged with (S)-5-(4-hydroxyphenyl)-3,4-dihydro-2H-pyrrole-2-carboxylic acid hydrochloride (133.7 g) and methanol (15V). To the resulting solution was added concentrated HCl (0.2V) and the solution was heated under reflux for about 18 h. The progress of the reaction was monitored for completion by HPLC. The solution was cooled to ambient temperature and concentrated in vacuo at 45° C. to near dryness. Acetone (9.7V) was charged, and the mixture was concentrated in vacuo to near dryness. Acetone (15V) was charged and the mixture was aged at ambient temperature for about 3 h. Solids were isolated by filtration, washed twice with acetone (2V) and dried to constant weight under a flow of nitrogen to afford 132.6 g (94%) of the title compound as a white solid.

### Example 4: Preparation of 1-(tert-butyl) 2-methyl (2S, 5R)-5-(4-hydroxyphenyl)pyrrolidine-1,2-dicarboxylate

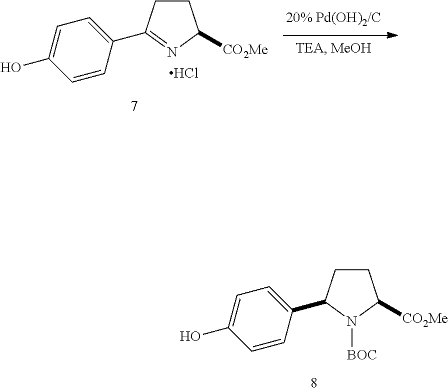 [SMILES: COC(=O)[C@@H]1CCC(c2ccc(O)cc2)=N1 ; Cl ; COC(=O)[C@@H]1CC[C@H](c2ccc(O)cc2)N1C(=O)OC(C)(C)C]

A hydrogenation vessel was charged with methyl (S)-5-(4-hydroxyphenyl)-3,4-dihydro-2H-pyrrole-2-carboxylate hydrochloride (66.9 g), 20% Pd(OH)₂/C (4 wt %) and MeOH (14.9V). Triethylamine (1.0 eq) was added over 5-10 min with good agitation, and the resulting suspension was stirred for an additional 5 min at ambient temperature. A solution of di-tert-butyl dicarbonate (1.0 eq) in MeOH (3V) was added and the container was rinsed forward into the hydrogenation vessel with additional MeOH (2.1V). The mixture was stirred under 1 atm hydrogen gas pressure for about 4.5 h. The progress of the reaction was monitored by HPLC.

The catalyst was removed by filtration through a pad of Celite and the reactor and spent filter cake were washed thrice with MeOH (3V). The filtrate was concentrated in vacuo to near dryness.

A second reaction was performed using 64.8 g of methyl (S)-5-(4-hydroxyphenyl)-3,4-dihydro-2H-pyrrole-2-carboxylate hydrochloride.

The MeOH filtrates from both runs were combined and the resulting solution was concentrated in vacuo at 42° C. to a thick paste. Fresh MeOH (4V) was charged and the mixture was stirred at ambient temperature for about 30 min. Water (IV) was charged over about 15 min, the mixture was aged at ambient temperature for about 18 h, cooled to and aged at 0-5° C. for about 3.5 h and filtered. The filter cake was washed thrice with cold 1:3 (v/v) MeOH-water (1V) and dried to constant weight under a flow of nitrogen to afford 134.1 g (81%) of the title compound as a white solid.

### Example 5: Preparation of (4-((2-fluorobenzyl)oxy)phenyl)boronic acid

Procedure A

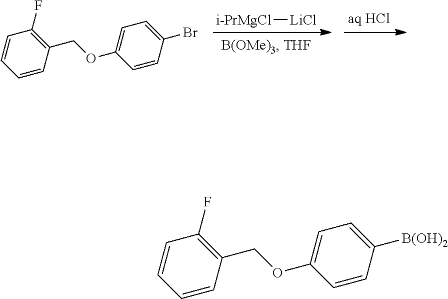

A nitrogen flushed reactor was charged with 1-[(4-bromophenoxy)methyl]-2-fluorobenzene (240 g) and anhydrous THF (4V). The resulting solution was cooled to −5 C° and commercial i-PrMgCl—LiCl/THF solution (1.2 eq; Sigma Aldrich) was added over about 45 min with control of the temperature below 0° C. The contents of the reactor was warmed to ambient temperature over about 3 h and was stirred overnight under an inert atmosphere of nitrogen. The contents of the reactor were cooled to −5° C. and B(OMe)₃ (2 eq) was added over about 30 min with control of the temperature below 0° C. The contents of the reactor were warmed to ambient temperature over about 90 min, aged for about 2 h and re-cooled to a temperature of −5° C. 1M HCl solution (4V) was added with control of the temperature below 5° C. Following the addition, water (6V) was added and the layers were separated. The layer was back extracted with EtOAc (4V) and the combined organic layers were washed with brine (10V) and concentrated to dryness in vacuo. Heptanes (5V) was added and the mixture was heated to and maintained at 65° C. for about 1 h. The contents of the flask were cooled to ambient temperature, then to 0-5° C. and were aged for about 1 h. The solids were collected by filtration, washed with cold heptane (2V) and dried to constant weight in vacuo at ambient temperature to yield 118 g (56%) of the title compound as a white solid.

A second experiment performed at the same scale gave 111 g (53%) of the title compound as a white solid.

Procedure B

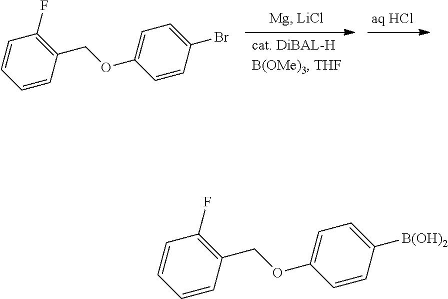 [SMILES: Fc1ccccc1COc1ccc(Br)cc1 ; OB(O)c1ccc(OCc2ccccc2F)cc1]

A nitrogen flushed reactor was charged with magnesium turnings (2.5 eq) and commercial LiCl/THF solution (1.25 eq, Sigma Aldrich). After adding commercial DIBAL-H/THF solution (0.01 eq), the contents of the reactor were stirred for about 15 min at ambient temperature under an inert atmosphere of nitrogen. A solution of 1-[(4-bromophenoxy)methyl]-2-fluorobenzene (44 g) in anhydrous THF (2V) was added dropwise, and the contents of the reactor were stirred for about 24 h at ambient temperature. The contents of the reactor were cooled to −5° C. and B(OMe)₃ (2 eq) was added with control of the temperature below 0° C. The contents of the reactor were aged for about 1 h at 0° C. and 0.1M HCl solution (10V) was added. The mixture was stirred for about 5 min, isopropanol (2V) was added and the mixture was aged for about 15 min at 0° C. The solids were collected by filtration, washed with water (2V) and dried to constant weight in vacuo at 40° C. to yield 24.2 g (62%) of the title compound as a white solid.

### Example 6: Preparation of (S)-3-(3-(tert-butoxycarbonyl)-5-oxooxazolidin-4-yl)propanoic acid

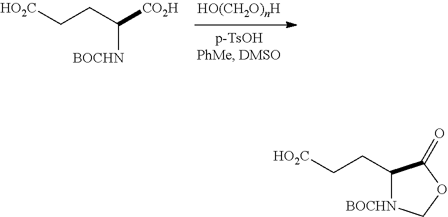

A reactor was charged with Boc-Glu (250 g), paraformaldehyde (0.5 wt %), p-TsOH (10 mol %), toluene (10V) and DMSO (1V). The reaction was heated to reflux and aged for about 1 h. Thereafter, the reaction was continued at reflux for about 3 h with distillate collection in a Dean-Stark trap. The reaction was cooled to ambient temperature, and water (5V) and MTBE (10V) were charged. The phases were separated and the organic layer was washed twice with brine (8V). The organic layer was concentrated to a minimum volume in vacuo. To the resulting residue was charged 1:1 (v/v) MTBE-heptane (5V) and product seed (1 g). The mixture was stirred for about 1 h at ambient temperature. Solids were collected by filtration and washed with ice cold 1:1 (v/v) MTBE-heptane (0.5V). The filtrate was concentrated to a minimum volume in vacuo. To the resulting residue was charged 1:1 (v/v) MTBE-heptane (2.5V). The mixture was stirred for about 1 h at ambient temperature. Solids were collected by filtration and washed with ice cold 1:1 (v/v) MTBE-heptane (0.5V). The solids from both filtrations were combined and dried to constant weight to give 143 g (54%) of the title product.

A second experiment performed at the same scale gave 139 g (53%) of the title product.

### Example 7: Preparation of (S)-4-((tert-butoxycarbonyl)amino)-5-methoxy-5-oxopentanoic pivalic anhydride

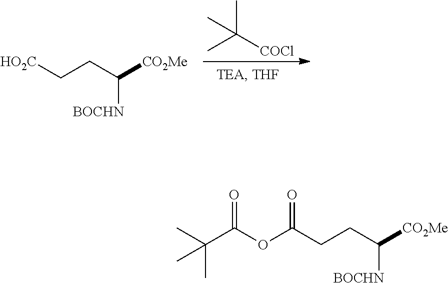

A reactor was charged with Boc-Glu-OMe (2 g) and THF (10V). After cooling to about 0° C., TEA (1.1 eq) was charged, followed by dropwise addition of pivaloyl chloride (1.1 eq). The reaction was stirred for about 2 h at 0° C. Solids were removed by filtration and the filtrate was concentrated in vacuo to give 2.57 g (97%) of the title compound as a clear oil which was used without further purification.

### Example 8: Preparation of (S)-3-(3-(tert-butoxycarbonyl)-5-oxooxazolidin-4-yl)propanoic pivalic anhydride

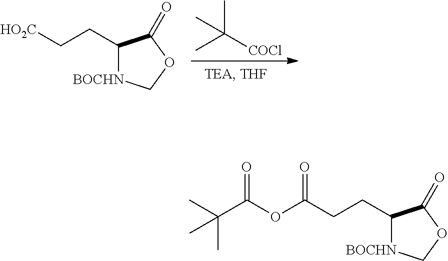

A reactor was charged with (S)-3-(3-(tert-butoxycarbonyl)-5-oxooxazolidin-4-yl)propanoic acid (10.2 g) and THF (10V). After cooling to about 0° C., TEA (1.1 eq) was charged, followed by dropwise addition of pivaloyl chloride (1.1 eq). The reaction was stirred for about 2 h at 0° C. then cooled to about −10° C. and aged for about 30 min. Solids were removed by filtration and the filtrate was concentrated in vacuo. The resulting oil was twice triturated with heptane (40 mL) The heptane layers were combined and concentrated in vacuo to give 7.91 g (59%) of the title compound.

### Example 9: Preparation of 1-methyl 5-(pyridin-2-yl) (tert-butoxycarbonyl)-L-glutamate

Procedure A

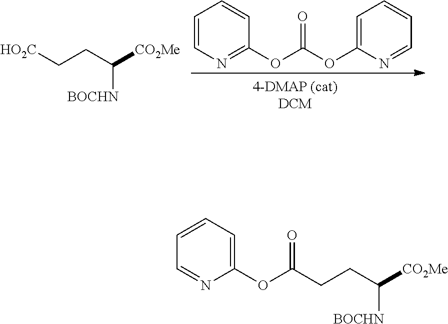

A reactor was charged with Boc-Glu-OMe (0.5 g) and DCM (10V). After cooling to about 0° C., di-(2-pyridyl)carbonate (1.1 eq) and 4-DMAP (0.05 eq) were added, and the reaction was aged for about 1 h. The reaction was washed with ice cold saturated NaHCO₃ solution and concentrated to dryness. The resulting residue was purified by column chromatography to give 473 mg (73%) of the title compound.

Procedure B

A reactor was charged with Boc-Glu-OMe (3.0 g) and DCM (10V). After cooling to about 0° C., di-(2-pyridyl)carbonate (1.1 eq) and 4-DMAP (0.05 eq) were added, and the reaction was aged for about 1 h. The reaction was washed with ice cold saturated NaHCO₃ solution and concentrated to dryness to give 3.5 g (90%) of the title compound as an oil which was used without further purification.

Procedure C

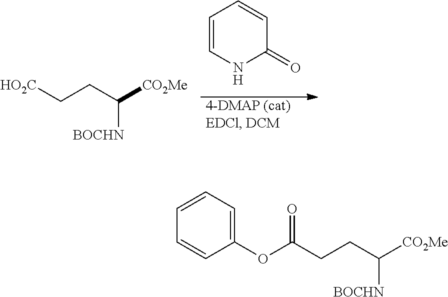

A reactor was charged with Boc-Glu-OMe (32 g) and DCM (10V). After adding 2-pyridone (1.2 eq), EDCI (1.1 eq) and 4-DMAP (0.1 eq), the reaction was aged for about 4 h. The reaction was quenched with water (10V), the layers were separated, the organic layer was washed with ice cold saturated NaHCO₃ solution and concentrated to dryness in vacuo. The resulting residue was purified by column chromatography to give 27 g (77%) of the title compound as a colorless oil.

### Example 10: Preparation of methyl (S)-5-(4-(benzyloxy)phenyl)-2-((tert-butoxycarbonyl)amino)-5-oxopentanoate

Procedure A

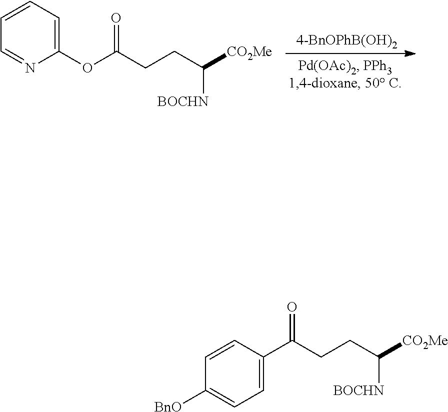

A reactor was charged with 1-methyl 5-(pyridin-2-yl) (tert-butoxycarbonyl)-L-glutamate (3.47 g (Example 9, Procedure B)), 4-BnOPhB(OH)₂ (2 eq), Pd(OAc)₂ (3 mol %), PPh₃ (9 mol %) and 1,4-dioxane (2V). The reaction was heated to 50° C.; the progress of the reaction was monitored for completion (HPLC). After about 16 h, the reaction was cooled to ambient temperature and was concentrated to dryness in vacuo. The residue was purified by column chromatography to give 1.34 g (35%) of the title compound.

Procedure B

A reactor was charged with 1-methyl 5-(pyridin-2-yl) (tert-butoxycarbonyl)-L-glutamate (16.5 g (Example 9, Procedure C)), 4-BnOPhB(OH)₂ (2 eq), Pd(OAc)₂ (5 mol %), PPh₃ (15 mol %) and 1,4-dioxane (2V). The reaction was heated to 50° C.; the progress of the reaction was monitored for completion (HPLC). After about 16 h, the reaction was cooled to ambient temperature and was concentrated to dryness in vacuo. The residue was dissolved in EtOAc and washed with saturated NaHCO₃ solution. The organic layer was concentrated to dryness in vacuo and the resulting residue was purified by column chromatography to give 12.6 g (69%) of the title compound as a white solid.

### Example 11: Preparation of methyl (S)-5-(4-(benzyloxy)phenyl)-2-((tert-butoxycarbonyl)amino)-5-oxopentanoate

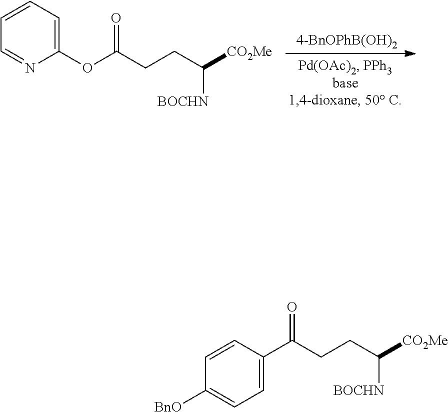

Procedure A

A reactor was charged with 1-methyl 5-(pyridin-2-yl) (tert-butoxycarbonyl)-L-glutamate (100 mg (non-chromatographed)), 4-BnOPhB(OH)₂ (2 eq), Pd(OAc)₂ (3 mol %), triphenylphosphine (9 mol %), KHCO₃ (2.5 eq) and 1,4-dioxane (2V). The reaction was maintained at 50° C. for 8 h. The reaction was cooled to ambient temperature and was concentrated to dryness in vacuo. The residue was analyzed and shown to contain 3.1% area (HPLC; 218 nm) of the title compound

Procedure B

A reactor was charged with 1-methyl 5-(pyridin-2-yl) (tert-butoxycarbonyl)-L-glutamate (100 mg (non-chromatographed)), 4-BnOPhB(OH)₂ (2 eq), Pd(OAc)₂ (3 mol %), triphenylphosphine (9 mol %), KH₂PO₄ (2.5 eq) and 1,4-dioxane (2V). The reaction was maintained at 50° C. for 8 h. The reaction was cooled to ambient temperature and was concentrated to dryness in vacuo. The residue was analyzed and shown to contain 16.0% area (HPLC; 218 nm) of the title compound.

Procedure C

A reactor was charged with 1-methyl 5-(pyridin-2-yl) (tert-butoxycarbonyl)-L-glutamate (100 mg (non-chromatographed)), 4-BnOPhB(OH)₂ (2 eq), Pd(OAc)₂ (3 mol %), triphenylphosphine (9 mol %), NH₄HCO₃ (2.5 eq) and 1,4-dioxane (2V). The reaction was maintained at 50° C. for 8 h. The reaction was cooled to ambient temperature and was concentrated to dryness in vacuo. The residue was analyzed and shown to contain 1.7% area (HPLC; 218 nm) of the title compound.

Procedure D

A reactor was charged with 1-methyl 5-(pyridin-2-yl) (tert-butoxycarbonyl)-L-glutamate (100 mg (non-chromatographed)), 4-BnOPhB(OH)₂ (2 eq), Pd(OAc)₂ (3 mol %), triphenylphosphine (9 mol %), KOAc (2.5 eq) and 1,4-dioxane (2V). The reaction was maintained at 50° C. for 8 h. The reaction was cooled to ambient temperature and was concentrated to dryness in vacuo. The residue was analyzed and shown to contain 1.4% area (HPLC; 218 nm) of the title compound.

### Example 12: Preparation of methyl (S)-5-(4-(benzyloxy)phenyl)-2-((tert-butoxycarbonyl)amino)-5-oxopentanoate

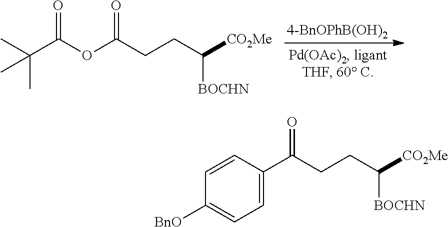

Procedure A

A reactor was charged with (S)-4-((tert-butoxycarbonyl)amino)-5-methoxy-5-oxopentanoic pivalic anhydride (100 mg), 4-BnOPhB(OH)₂ (2 eq), Pd(OAc)₂ (5 mol %), PPh₃ (15 mol %) and THF (40V). The reaction was maintained at 60° C. for 24 h. The reaction was cooled to ambient temperature and 1,3,5-trimethoxybenzene (1 eq; internal standard) was charged, and the solution was concentrated to dryness in vacuo. The residue was analyzed (NMR) and shown to contain a 31% yield of the title compound.

Procedure B

A reactor was charged with (S)-4-((tert-butoxycarbonyl)amino)-5-methoxy-5-oxopentanoic pivalic anhydride (100 mg), 4-BnOPhB(OH)₂ (2 eq), Pd(OAc)₂ (5 mol %), (p-MeOPh)₃P (15 mol %) and THF (40V). The reaction was maintained at 60° C. for 24 h. The reaction was cooled to ambient temperature and 1,3,5-trimethoxybenzene (1 eq; internal standard) was charged, and the solution was concentrated to dryness in vacuo. The residue was analyzed (NMR) and shown to contain a 30% yield of the title compound.

Procedure C

A reactor was charged with (S)-4-((tert-butoxycarbonyl)amino)-5-methoxy-5-oxopentanoic pivalic anhydride (100 mg), 4-BnOPhB(OH)₂ (2 eq), Pd(OAc)₂ (5 mol %), (cyclohexyl)₃P (15 mol %) and THF (40V). The reaction was maintained at 60° C. for 24 h. The reaction was cooled to ambient temperature and 1,3,5-trimethoxybenzene (1 eq; internal standard) was charged, and the solution was concentrated to dryness in vacuo. The residue was analyzed (NMR) and shown to contain a 21% yield of the title compound.

### Example 13: Preparation of ter-butyl (S)-4-(3-(4-(benzyloxy)phenyl)-3-oxopropyl)-5-oxooxazolidine-3-carboxylate

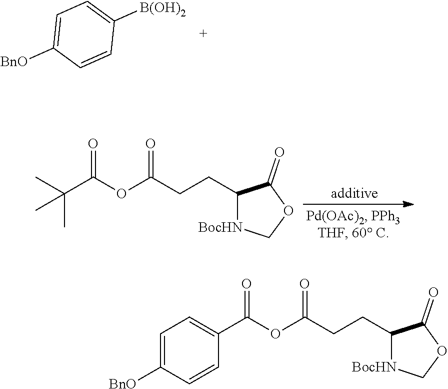

Procedure A

A reactor was charged with (S)-3-(3-(tert-butoxycarbonyl)-5-oxooxazolidin-4-yl)propanoic pivalic anhydride (100 mg), Pd(OAc)₂ (5 mol %), PPh₃ (15 mol %), K2CO₃ (2 eq) and THF (40V). The reaction was maintained at 60° C. for 24 h. The reaction was cooled to ambient temperature and 1,3,5-trimethoxy benzene (1 eq; internal standard) was charged, and the solution was concentrated to dryness in vacuo. The residue was analyzed (NMR) and was shown to contain a 6% yield of the title compound.

Procedure B

A reactor was charged with (S)-3-(3-(tert-butoxycarbonyl)-5-oxooxazolidin-4-yl)propanoic pivalic anhydride (100 mg), Pd(OAc)₂ (5 mol %), PPh₃ (15 mol %), water (2 eq) and THF (40V). The reaction was maintained at 60° C. for 24 h. The reaction was cooled to ambient temperature and 1,3,5-trimethoxybenzene (1 eq; internal standard) was charged, and the solution was concentrated to dryness in vacuo. The residue was analyzed (NMR) and was shown to contain a 7% yield of the title compound.

### Example 14: Preparation of tert-butyl (S)-4-(3-(4-(benzyloxy)phenyl)-3-oxopropyl)-5-oxooxazolidine-3-carboxylate

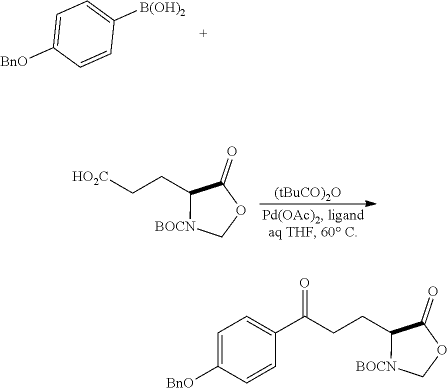

Procedure A

A reactor was charged with (S)-3-(3-(tert-butoxycarbonyl)-5-oxooxazolidin-4-yl)propanoic acid (0.6 g), Pd(OAc)₂ (3 mol %), dppf (4 mol %) and THF (4V). After charging a solution of 4-BnOPhB(OH)₂ (1.2 eq) in THF (2V), pivalic anhydride (1.5 eq) and water (2.5 eq) were added, and the reaction was maintained at 60° C. for 16 h. The reaction was cooled to ambient temperature and was concentrated to dryness in vacuo. The residue was purified by column chromatography to give 128 mg (13%) of the title compound as a white solid.

Procedure B

A reactor was charged with (S)-3-(3-(tert-butoxycarbonyl)-5-oxooxazolidin-4-yl)propanoic acid (1.0 g), Pd(OAc)₂ (5 mol %), PPh₃ (10 mol %), 4-BnOPhB(OH)₂ (1.2 eq), pivalic anhydride (1.5 eq), water (2.5 eq) and THF (2V). The reaction was stirred at ambient temperature for 1 h followed by heating to 60° C. for 24 h. The reaction was cooled to ambient temperature and quenched by addition of saturated NaHCO₃ solution. The reaction was extracted twice with EtOAc (10V) and the combined organic layers were filtered through a pad of Celite®/silica gel, the spent pad was washed with EtOAc and the filtrate was concentrated to dryness in vacuo. The residue was purified by column chromatography to give 1.13 g (69%) of the title compound.

### Example 15: Preparation of tert-butyl (S)-4-(3-(4-hydroxyphenyl)-3-oxopropyl)-5-oxooxazolidine-3-carboxylate

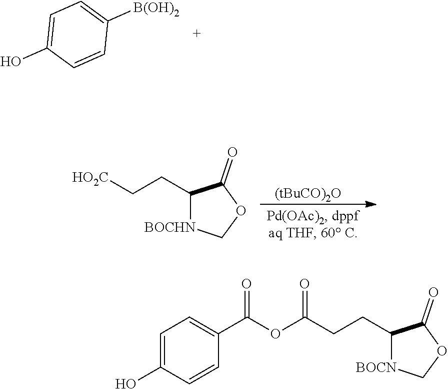

A reactor was charged with (S)-3-(3-(tert-butoxycarbonyl)-5-oxooxazolidin-4-yl)propanoic acid (1.0 g), (4-hydroxyphenyl)bornonic acid (1.2 eq), Pd(OAc)₂ (5 mol %), PPh₃ (15 mol %), pivalic anhydride (1.5 eq), water (2.5 eq) and THF (3V). After stirring for 1 h at ambient temperature, the reaction was heated to and maintained at 60° C. for 24 h. The reaction was cooled to ambient temperature and EtOAc (20V) and saturated NaHCO₃ solution (10V) were added. The layers were separated and the organic layer was washed with brine (10V). The organic layer was dried, filtered and concentrated to dryness in vacuo. The residue was purified by column chromatography to give 334 mg (26%) of the title compound.

### Example 16: Preparation of methyl (S)-5-(4-(benzyloxy)phenyl)-3,4-dihydro-2H-pyrrole-2-carboxylate

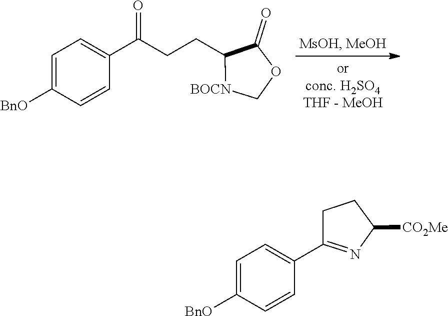

Procedure A

A reactor was charged with tert-butyl (S)-4-(3-(4-(benzyloxy)phenyl)-3-oxopropyl)-5-oxooxazolidine-3-carboxylate (1 g) and MeOH (5V). After charging MsOH (5V), the reaction was stirred at ambient temperature for 6 h. The reaction was neutralized with saturated NaHCO₃ solution, extracted with EtOAc and the organic layer was concentrated to dryness in vacuo to give 680 mg (93%) of the title compound as a white solid.

Procedure B

A reactor was charged with tert-butyl (S)-4-(3-(4-(benzyloxy)phenyl)-3-oxopropyl)-5-oxooxazolidine-3-carboxylate (600 mg), THF (10V) and MeOH (5V) and the solution was cooled to about 0° C. After charging concentrated H₂SO₄ (5V), the reaction was stirred for 3 h.

The reaction was neutralized with saturated NaHCO₃ solution, extracted with EtOAc and the organic layer was concentrated to dryness in vacuo. The resulting residue was dissolved in 1:2 (v/v) IPA-water (10V) at 40° C., the solution was cooled to 0-5° C. and aged for about 15 min. Solids were isolated by filtration and dried in vacuo at 40° C. to give 287 mg (66%) of the title compound as a white solid.

### Example 17: Preparation of methyl (S)-5-(4-hydroxyphenyl)-3,4-dihydro-2H-pyrrole-2-carboxylate

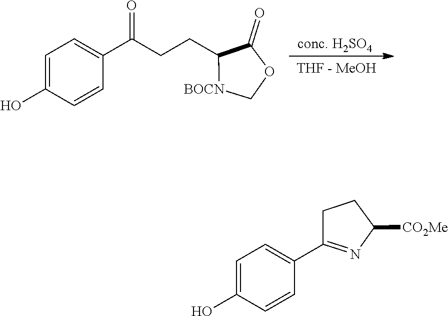

A reactor was charged with tert-butyl (S)-4-(3-(4-hydroxyphenyl)-3-oxopropyl)-5-oxooxazolidine-3-carboxylate (200 mg) and THF (10V). After charging concentrated H₂SO₄ (5V), the reaction was stirred at ambient temperature for 2 h. The reaction was neutralized with saturated NaHCO₃ solution, extracted twice with EtOAc (50V) and the combined organic layers were concentrated to dryness in vacuo to give 119 mg (91%) of the title compound.

### Example 18: Preparation of (S)-5-(4-(benzyloxy)phenyl)-3,4-dihydro-2H-pyrrole-2-carboxamide

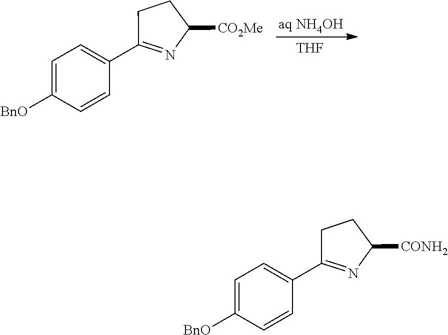 [SMILES: COC(=O)[C@@H]1CCC(c2ccc(OCc3ccccc3)cc2)=N1 ; NC(=O)[C@@H]1CCC(c2ccc(OCc3ccccc3)cc2)=N1 ; "THF"]

A reactor was charged with methyl (S)-5-(4-(benzyloxy)phenyl)-3,4-dihydro-2H-pyrrole-2-carboxylate (100 mg), THF (1V) and NH₄OH (4V) and the reaction was stirred at 60° C. for 5 h. The reaction was concentrated to dryness in vacuo, and water (10V) and EtOAc (20V) were added to the residue. The layers were separated, and the organic layer was dried, filtered and concentrated in vacuo to give 89 mg (94%) of the title compound as a yellow solid.

### Example 19: Preparation of ter-butyl (S)-(1-amino-5-(4-(benzyloxy)phenyl)-1,5-dioxopentan-2-yl)carbamate

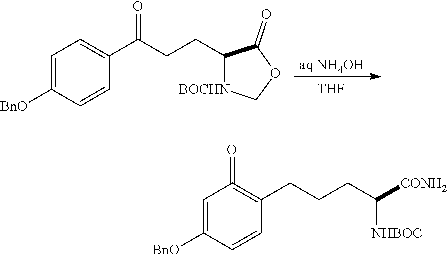

Procedure A

A reactor was charged with tert-butyl (S)-4-(3-(4-(benzyloxy)phenyl)-3-oxopropyl)-5-oxooxazolidine-3-carboxylate (200 mg), THF (V) and NH₄OH (4V) and the reaction was stirred at ambient temperature for 36 h. The reaction was neutralized with 1M HCl to about pH 3 and was extracted with EtOAc. The organic layer was dried, filtered and concentrated in vacuo to give 139 mg (72%) of the title compound as an off-white solid.

Procedure B

A vial reactor was charged with tert-butyl (S)-4-(3-(4-(benzyloxy)phenyl)-3-oxopropyl)-5-oxooxazolidine-3-carboxylate (200 mg), THF (1V) and NH₄OH (4V) and the reaction was stirred at 60° C. for 1 h. The progress of the reaction was monitored for completion (HPLC). The solution was cooled to ambient temperature, additional NH₄OH (2V) was added and the reaction was stirred at 60° C. for 1 h. The reaction was neutralized with 1M HCl to about pH 3 and was extracted with EtOAc. The organic layer was dried, filtered and concentrated in vacuo to give 157 mg (81%) of the title compound as an off-white solid.

Procedure C

A reactor was charged with tert-butyl (S)-4-(3-(4-(benzyloxy)phenyl)-3-oxopropyl)-5-oxooxazolidine-3-carboxylate (2 g), THF (1V) and NH₄OH (4V) and the reaction was stirred at ambient temperature for 36 h. The reaction was concentrated to dryness in vacuo, and the residue was stirred with MTBE (7.5V) for about 15 min. Solids were filtered and dried in vacuo to give 1.86 g (96%) of the title compound as an off-white solid.

### Example 20: Preparation of(S)-5-(4-(benzyloxy)phenyl)-3,4-dihydro-2H-pyrrole-2-carboxamide

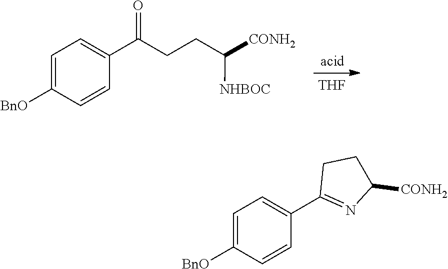

Procedure A

A reactor was charged with tert-butyl (S)-(1-amino-5-(4-(benzyloxy)phenyl)-1,5-dioxopentan-2-yl)carbamate (100 mg), THF (IV) and MsOH (4V) and the reaction was stirred at ambient temperature for 36 h. The reaction was neutralized with saturated NaHCO₃ to about pH 8 and was extracted with EtOAc (20V). The organic layer was dried, filtered and concentrated in vacuo. The resulting residue was purified by column chromatography to give 39 mg (55%) of the title compound as a yellow solid.

Procedure B

A reactor was charged with tert-butyl (S)-(1-amino-5-(4-(benzyloxy)phenyl)-1,5-dioxopentan-2-yl)carbamate (1.0 g) and THF (10V). After cooling to about 0° C., concentrated HCl (20V) was added and the reaction was stirred at ambient temperature for 1 h. The reaction was neutralized with saturated NaHCO₃ to about pH 8 and the THF was removed by distillation in vacuo. Solids were filtered and dried in vacuo to give 579 mg (81%) of the title compound as a yellow solid.

### Example 21: Preparation of methyl (S)-5-(4-(benzyloxy)phenyl)-3,4-dihydro-2H-pyrrole-2-carboxylate

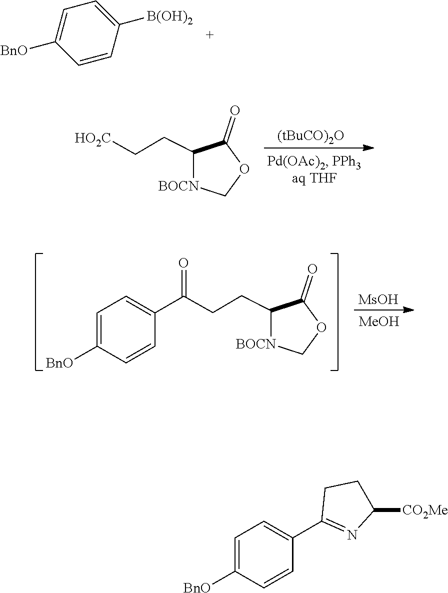

### Part A: in situ preparation of tert-butyl (S)-4-(3-(4-(benzyloxy)phenyl)-3-oxopropyl)-5-oxooxazolidine-3-carboxylate

A reactor was charged with (S)-3-(3-(tert-butoxycarbonyl)-5-oxooxazolidin-4-yl)propanoic acid (5 g), 4-BnOPhB(OH)₂ (1.2 eq), Pd(OAc)₂ (5 mol %), PPh₃ (15 mol %), pivalic anhydride (1.5 eq), water (2.5 eq) and THF (2V). The reaction was stirred at ambient temperature for 1 h followed by heating to 60° C. and holding for 24 h. The reaction was cooled to ambient temperature to give a THF solution of the title compound.

### Part B: Preparation of methyl (S)-5-(4-(benzyloxy)phenyl)-3,4-dihydro-2H-pyrrole-2-carboxylate

To the THF solution from Part A was charged MeOH (2V) and MsOH (2V) and the reaction was stirred at ambient temperature for 6 h. The reaction was quenched by addition of saturated NaHCO₃ solution and was twice extracted with EtOAc (10V). The combined organic layers were filtered through a pad of Celite®/silica gel, the spent pad was washed with EtOAc and the filtrate was concentrated to dryness in vacuo. The resulting residue was purified by column chromatography to give 3.74 g (63%) of the title compound.

### Example 22: Preparation of tert-butyl (S)-(1-amino-5-(4-(benzyloxy)phenyl)-1,5-dioxopentan-2-yl)carbamate

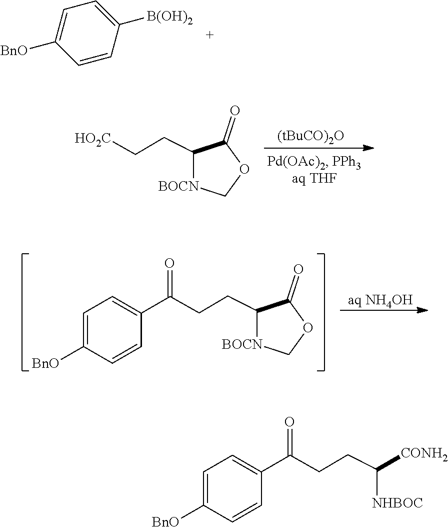

### Part A: in situ preparation of tert-butyl (S)-4-(3-(4-(benzyloxy)phenyl)-3-oxopropyl)-5-oxooxazolidine-3-carboxylate

A reactor was charged with (S)-3-(3-(tert-butoxycarbonyl)-5-oxooxazolidin-4-yl)propanoic acid (5 g), 4-BnOPhB(OH)₂ (1.2 eq), Pd(OAc)₂ (5 mol %), PPh₃ (15 mol %), pivalic anhydride (1.5 eq), water (2.5 eq) and THF (10V). The reaction was stirred at ambient temperature for 1 h followed by heating to 60° C. and holding for 24 h. The reaction was cooled to ambient temperature to give a THF solution of the title compound.

### Part B: Preparation of tert-butyl (S)-(1-amino-5-(4-(benzyloxy)phenyl)-1,5-dioxopentan-2-yl)carbamate

To the THF solution from Part A was charged NH₄OH solution (10V), the reaction was stirred at ambient temperature for 8 h followed by heating to 60° C. and holding for 3 h. The reaction was cooled to ambient temperature and EtOAc (20V) and water (10V) were charged. The layers were separated and the organic layer was washed with brine. The organic layer was filtered through a pad of Celite®/silica gel, the spent pad was washed with EtOAc (10V) and the filtrate was dried, filtered and concentrated to dryness in vacuo. To the resulting residue was added MTBE (30V), the mixture was heated to and held at reflux for about 15 min, cooled to ambient temperature, then to 0-5° C. and aged for about 15 min. Solids were isolated by filtration and washed twice with ice cold MTBE (4V). The resulting solids were slurried in heptane (20V) and the mixture was heated to reflux and aged for about 15 min. After cooling to ambient temperature, the slurry was further cooled to 0-5° C. and aged for about 15 min. The solids were filtered, twice washed with heptane (4V) and dried to constant weight in vacuo to give 5.43 g (68%) of the title compound as a grey solid.

### Example 23: Preparation of(S)-5-(4-(benzyloxy)phenyl)-3,4-dihydro-2H-pyrrole-2-carboxamide

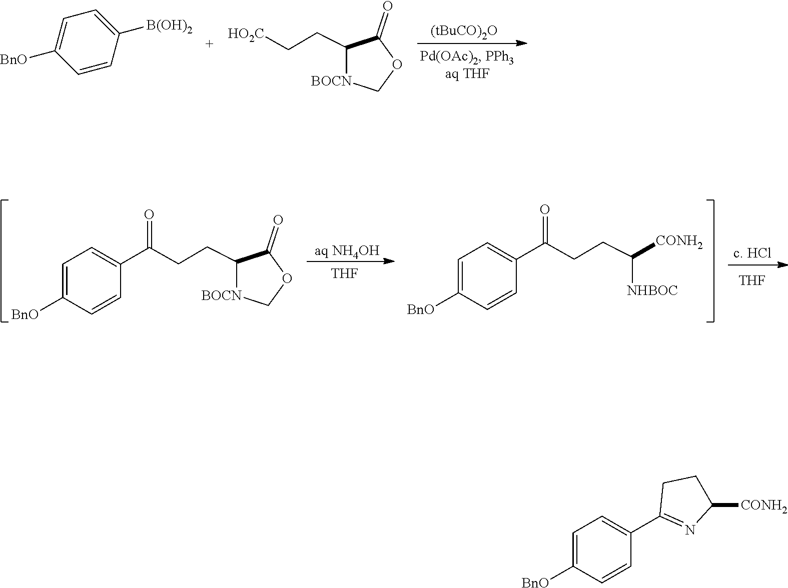

### Part A: In Situ Preparation of tert-butyl (S)-4-(3-(4-(benzyloxy)phenyl)-3-oxopropyl)-5-oxooxazolidine-3-carboxylate

A nitrogen flushed 1 L reactor was charged with (S)-3-(3-(tert-butoxycarbonyl)-5-oxooxazolidin-4-yl)propanoic acid (25 g), (4-(benzyloxy)phenyl)boronic acid (1.3 eq), PPh₃ (15 mol %), Pd(OAc)₂ (5 mol %), water (2.5 eq) and THF (6V) under an inert atmosphere of nitrogen. After stirring for about 5 min at ambient temperature, pivalic anhydride (1.5 eq) and water (2.5 eq) were charged, and after stirring an additional 5 min, the mixture was sparged with nitrogen for about 5 min. The mixture was aged for about 1 h, heated to 58-60° C. (reaction temperature) over about 1 h and aged for about 24 h. The progress of the reaction was monitored for completion (HPLC). The reaction mixture was cooled to room temperature under an inert atmosphere of nitrogen to give a THF solution of the title compound.

### Part B: In Situ Preparation of tert-butyl (S)-(1-amino-5-(4-(benzyloxy)phenyl)-1,5-dioxopentan-2-yl)carbamate

The THF solution from Part A was cooled to about 0° C. 28 wt % NH₄OH solution (10V) was added with good agitation while maintaining a reaction temperature of <5° C. The resulting mixture was warmed to ambient temperature over about 1 h and was aged for an additional 48 h; the progress of the reaction was monitored for completion (HPLC). The mixture was concentrated in vacuo at 20-25° C. for about 3 h followed by sparging with nitrogen for about 2 h to afford an solution of the title compound.

### Part C: Preparation of (S)-5-(4-((2-fluorobenzyl)oxy)phenyl)-3,4-dihydro-2H-pyrrole-2-carboxamide

To the solution from Part B was added THF (14V), the solution was aged for 1 h at ambient temperature, transferred into a 3 L reactor and cooled to about 0° C. over about 2 h. 37 wt % HCl (10V) was added with good agitation while maintaining a reaction temperature of <10° C. The mixture was warmed to ambient temperature over about 1 h and aged for about 2.5 h; the progress of the reaction was monitored for completion (HPLC). The reactor's contents were cooled to about 0° C. over about 2 h and water (14V) and EtOAc (20V) were charged. After stirring for about 5 min, the layers were separated and the layer was back extracted with EtOAc (20V). The layer was cooled to about 0° C. over about 1 h, and the pH was adjusted to ˜8 by addition of 10M aq NaOH solution. After charging EtOAc (20V) and stirring for about 30 min, the layers were separated and the layer was back extracted with EtOAc (10V). After stirring for about 15 min, the layers were separated and all EtOAc layers were combined and treated with aq NaOH (pH 10; 10V) for about 1 h. The layers were separated, the organic layer was passed through a pad of silica gel and Celite® and the spent pad was washed with EtOAc (4V). The filtrate was concentrated in vacuo at 50° C. to minimum volume, and the resulting residue was reconcentrated twice from MTBE (5V). MTBE (10V) was charged, the mixture was heated to reflux, aged about 1 h, cooled to room temperature over about 2 h, cooled to about 0° C. over 2 h and aged for an additional 2 h. The solids were isolated by filtration, washed with ice cold MTBE (2V) and dried in vacuo overnight at ambient temperature. The solids were stirred for about 18 h at ambient temperature after charging MTBE (9V) and IPA (1V). The mixture was cooled to about 0° C. over 2 h, filtered and washed with ice cold MTBE (2V). The solids were dried in vacuo overnight to give 11.8 g (42%) of the title compound as a white solid.

### Example 24: Preparation of tert-butyl (2S,5R)-2-carbamoyl-5-(4-hydroxyphenyl)pyrrolidine-1-carboxylate

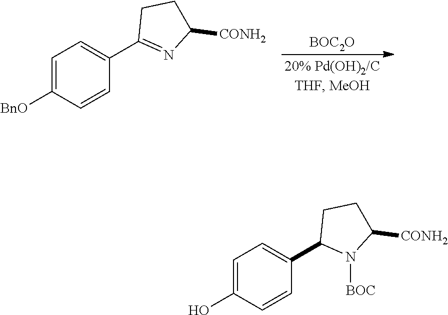

Procedure A

A reactor was charged with (S)-5-(4-(benzyloxy)phenyl)-3,4-dihydro-2H-pyrrole-2-carboxamide (300 mg), MeOH (10V), THF (10V), di-tert-butyldicarbonate (0.98 eq) and 20 wt % Pd(OH)₂/C (10 wt %) under an inert atmosphere of nitrogen. The mixture was stirred at ambient temperature under 1 atm hydrogen gas pressure; the progress of the reaction was monitored for completion (HPLC). After about 8 h, the mixture was filtered, and the filtrate was concentrated in vacuo. The resulting residue was purified by column chromatography to give 240 mg (77%) of the title compound as a yellow solid.

Procedure B

A reactor was charged with (S)-5-(4-(benzyloxy)phenyl)-3,4-dihydro-2H-pyrrole-2-carboxamide (11.5 g), MeOH (4.8V) and THF (10V). A solution of di-tert-butyldicarbonate (0.98 eq) in MeOH (4.8V) was charged, followed by 20 wt % Pd(OH)₂/C (10 wt/o) under an inert atmosphere of nitrogen. The mixture was stirred at ambient temperature under 1 atm hydrogen gas pressure; the progress of the reaction was monitored for completion (HPLC). After about 48 h, the mixture was filtered through a pad of Celite®, the spent pad was washed with 1:1 (v/v) THF-MeOH (4.8V), and the filtrate was concentrated in vacuo until no distillate was observed being collected. The resulting residue was reconcentrated in vacuo at 40° C. in triplicate from IPA (26V). The resulting residue was reconcentrated twice in vacuo after charging IPA (4.8V). To the residue was charged IPA (6.1V) and the mixture was aged for about 1 h at ambient temperature. Solids were isolated by filtration and dried in vacuo to constant weight. The solids were dissolved in 6:1 (v/v) IPA-water (6.1V) at 70° C., the solution was aged for about 2 h, cooled to ambient temperature over about 1 h and cooled to 0-5° C. over about 1 h and aged for about 3 h. Solids were isolated by filtration, washed with ice cold IPA (2V) and dried to constant weight in vacuo to give 4.74 g (43%) of the title compound.

### Example 25: Preparation of tert-butyl (2S,5R)-2-carbamoyl-5-(4-((2-fluorobenzyl)oxy)phenyl)pyrrolidine-1-carboxylate

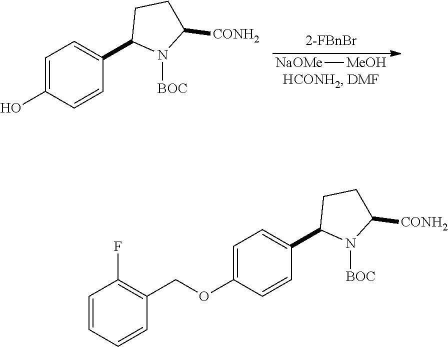

A reactor was charged with tert-butyl (2S,5R)-2-carbamoyl-5-(4-hydroxyphenyl)pyrrolidine-1-carboxylate (2.5 g), formamide (2.6V), DMF (3V) and 2-fluorobenzyl bromide (1.1 eq) under an inert atmosphere of nitrogen. The mixture was cooled to 15-20° C. and freshly titrated 25 wt % NaOMe-MeOH solution (1.05 eq) was added slowly over about 1 h. The progress of the reaction was monitored for completion (HPLC). Following an additional charge of 25 wt % NaOMe-MeOH solution (0.05 eq) and aging for about 100 min, the reaction was quenched by adding glacial acetic acid (1.15 eq) and water (1V). The mixture was heated to about 60° C., and water (3V) was added slowly over about 1 h. The mixture was cooled over about 40 min to ambient temperature, then to 0-5° C. The solids were collected by filtration, washed twice with water (2V) and dried to constant weight in vacuo at 80° C. to give 3.11 g (92%) of the title compound.

### Example 26: Preparation of methyl (S)-2-((tert-butoxycarbonyl)amino)-5-(4-((2-fluorobenzyl)oxy)phenyl)-5-oxopentanoate

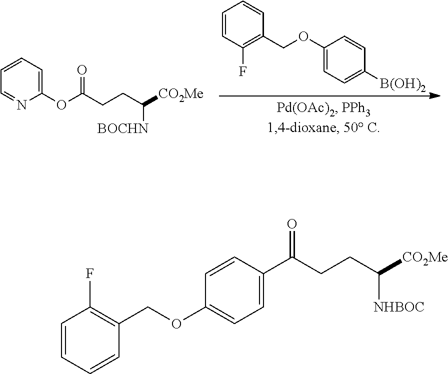

A reactor was charged with 1-methyl 5-(pyridin-2-yl) (tert-butoxycarbonyl)-L-glutamate (1.0 g (Example 9, Procedure C)), (4-((2-fluorobenzyl)oxy)phenyl)boronic acid (2 eq), Pd(OAc)₂ (5 mol %), PPh₃ (15 mol %) and 1,4-dioxane (2V). The reaction was heated to 50° C.; the progress of the reaction was monitored for completion (HPLC). After about 16 h, the reaction was cooled to ambient temperature and was concentrated to dryness in vacuo. The residue was dissolved in EtOAc and washed with saturated NaHCO₃ solution. The organic layer was concentrated to dryness in vacuo and the resulting residue was purified by column chromatography to give 847 mg (74%) of the title compound as a white solid.

### Example 27: Preparation of tert-butyl (S)-4-(3-(4-((2-fluorobenzyl)oxy)phenyl)-3-oxopropyl)-5-oxooxazolidine-3-carboxylate

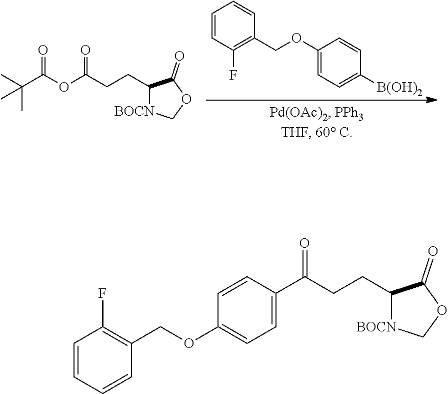

Procedure A

A reactor was charged with (S)-3-(3-(tert-butoxycarbonyl)-5-oxooxazolidin-4-yl)propanoic pivalic anhydride (900 mg), (4-((2-fluorobenzyl)oxy)phenyl)boronic acid (2 eq), Pd(OAc)₂ (5 mol %), PPh₃ (15 mol %) and THF (40V). The reaction was maintained at 60° C. for 24 h. The reaction was cooled to ambient temperature and concentrated to dryness in vacuo. The residue was purified by column chromatography to give 461 mg (40%) of the title compound as a white solid.

Procedure B

A reactor was charged with (S)-3-(3-(tert-butoxycarbonyl)-5-oxooxazolidin-4-yl)propanoic pivalic anhydride (1.52 g), (4-((2-fluorobenzyl)oxy)phenyl)boronic acid (1.2 eq), Pd(OAc)₂ (5 mol %), PPh₃ (15 mol %) and THF (20V). The reaction was maintained at 60° C. for 24 h. The reaction was cooled to ambient temperature and concentrated to dryness in vacuo. The residue was purified by column chromatography to give 1.52 g (77%) of the title compound as a white solid.

### Example 28: Preparation of tert-butyl (S)-4-(3-(4-((2-fluorobenzyl)oxy)phenyl)-3-oxopropyl)-5-oxooxazolidine-3-carboxylate

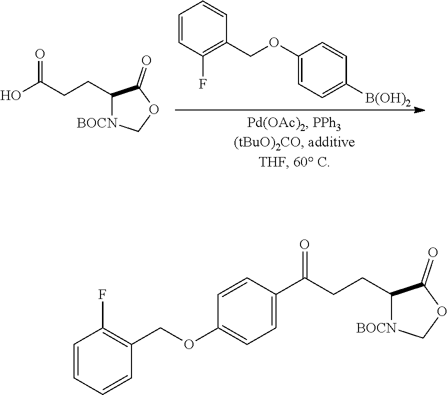

Procedure A

A reactor was charged with (S)-3-(3-(tert-butoxycarbonyl)-5-oxooxazolidin-4-yl)propanoic acid (100 mg), (4-((2-fluorobenzyl)oxy)phenyl)boronic acid (1.2 eq), Pd(OAc)₂ (5 mol %), PPh₃ (15 mol %), pivalic anhydride (1.5 eq) and THF (10V). The reaction was maintained at 60° C. for 24 h. The reaction was cooled to ambient temperature, 1,3,5-trimethoxybenzene (1 eq, internal standard) was added and the reaction was concentrated to dryness in vacuo. The residue was analyzed (NMR) and was shown to contain a 55% yield of the title compound.

Repetition of the experiment gave a residue shown to contain a 44% yield of the title compound (internal standard NMR analysis).

Procedure B

A reactor was charged with (S)-3-(3-(tert-butoxycarbonyl)-5-oxooxazolidin-4-yl)propanoic acid (100 mg), (4-((2-fluorobenzyl)oxy)phenyl)boronic acid (1.2 eq), Pd(OAc)₂ (5 mol %), PPh₃ (15 mol %), pivalic anhydride (1.5 eq), water (2.5 eq) and THF (10V). The reaction was maintained at ambient temperature for 1 h followed by heating to 60° C. for 24 h. The reaction was cooled to ambient temperature, 1,3,5-trimethoxybenzene (1 eq, internal standard) was added and the reaction was concentrated to dryness in vacuo. The residue was analyzed (NMR) and was shown to contain a 74% yield of the title compound.

Procedure C

A reactor was charged with (S)-3-(3-(tert-butoxycarbonyl)-5-oxooxazolidin-4-yl)propanoic acid (5 g), (4-((2-fluorobenzyl)oxy)phenyl)boronic acid (1.2 eq), Pd(OAc)₂ (5 mol %), PPh₃ (15 mol %), pivalic anhydride (1.5 eq), water (2.5 eq) and THF (10V). The reaction was maintained at ambient temperature for 1 h followed by heating to 60° C. for 24 h. The reaction was cooled to ambient temperature and the reaction was quenched by addition of saturated NaHCO₃ solution. The reaction was extracted twice with EtOAc (10V), the combined organic layers were filtered through a pad of Celite®/silica gel and the spent pad was washed with EtOAc. The filtrate was concentrated to dryness in vacuo and the resulting residue was purified by column chromatography to yield 6.83 g (80%) of the title compound.

### Example 29: Preparation of tert-butyl (S)-(1-amino-5-(4-((2-fluorobenzyl)oxy)phenyl)-1,5-dioxopentan-2-yl)carbamate

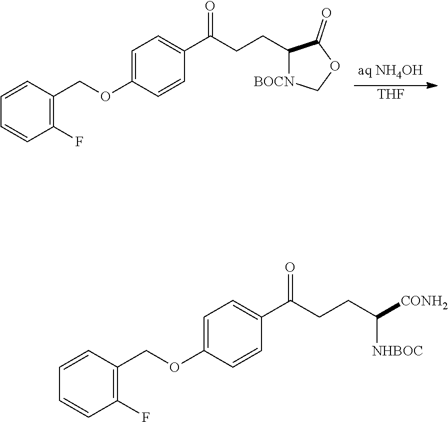

A reactor was charged with tert-butyl (S)-4-(3-(4-((2-fluorobenzyl)oxy)phenyl)-3-oxopropyl)-5-oxooxazolidine-3-carboxylate (100 mg), THF (10V) and NH₄OH solution (10V). The reaction was stirred at ambient temperature for 8 h then heated to and maintained at 60° C. for 3 h. The reaction was cooled to ambient temperature and solids were isolated by filtration.

The filtrate was concentrated to half volume and solids were isolated by filtration. The combined solids were dried to constant weight in vacuo at 40° C. to give 85 mg (88%) of the title compound as a white solid.

### Example 30: Preparation of methyl (S)-5-(4-((2-fluorobenzyl)oxy)phenyl)-3,4-dihydro-2H-pyrrole-2-carboxylate

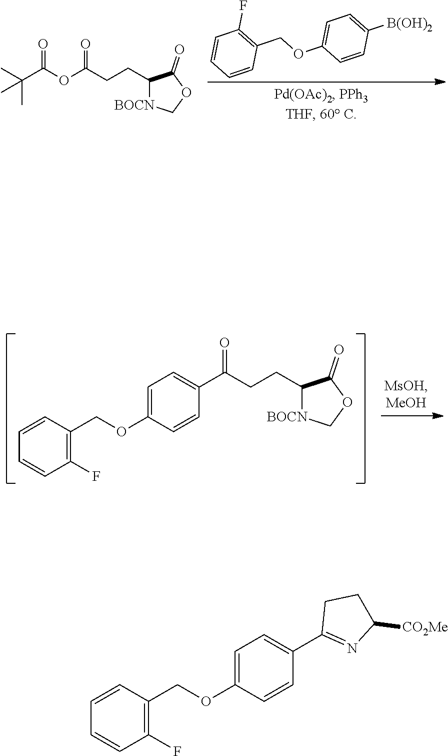

### Part A: In Situ Preparation of tert-butyl (S)-4-(3-(4-((2-fluorobenzyl)oxy)phenyl)-3-oxopropyl)-5-oxooxazolidine-3-carboxylate

A reactor was charged with (S)-3-(3-(tert-butoxycarbonyl)-5-oxooxazolidin-4-yl)propanoic pivalic anhydride (3.34 g), (4-((2-fluorobenzyl)oxy)phenyl)boronic acid (1.2 eq), Pd(OAc)₂ (5 mol %), PPh₃ (15 mol %) and THF (20V). The reaction was maintained at 60° C. for 24 h. The reaction was cooled to ambient temperature to give a THF solution containing the title compound.

### Part B: Preparation of methyl (S)-5-(4-((2-fluorobenzyl)oxy)phenyl)-3,4-dihydro-2H-pyrrole-2-carboxylate

To the THF solution from Part A was added MeOH (1V) and MsOH (1V), and the reaction was aged at ambient temperature for about 6 h. The progress of the reaction was monitored for completion (HPLC). Additional MeOH (1V) and MsOH (1V) were charged, and the reaction was aged at ambient temperature for about 8 h. The reaction was neutralized to about pH 8 using saturated NaHCO₃ solution. The reaction was extracted with EtOAc (10V), and the organic layer was concentrated to dryness in vacuo. The residue was purified by column chromatography to give 2.92 g (68%) of the title compound.

### Example 31: Preparation of tert-butyl (S)-(1-amino-5-(4-((2-fluorobenzyl)oxy)phenyl)-1,5-dioxopentan-2-yl)carbamate

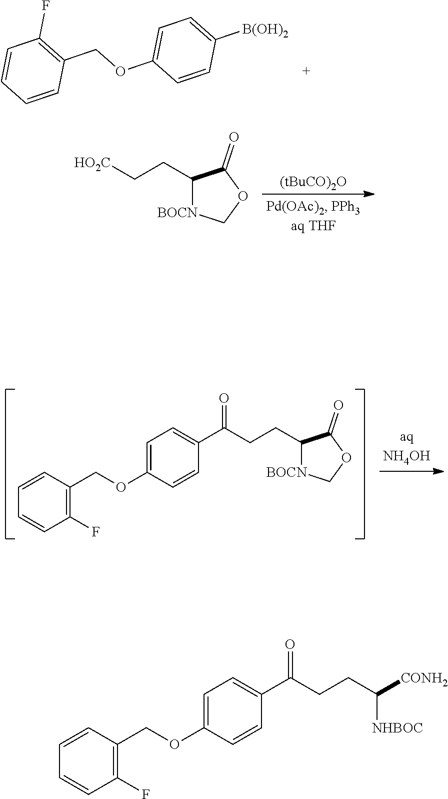

### Part A: In Situ Preparation of tert-butyl (S)-4-(3-(4-((2-fluorobenzyl)oxy)phenyl)-3-oxopropyl)-5-oxooxazolidine-3-carboxylate

A reactor was charged with (S)-3-(3-(tert-butoxycarbonyl)-5-oxooxazolidin-4-yl)propanoic acid (5 g), (4-((2-fluorobenzyl)oxy)phenyl)boronic acid (1.2 eq), Pd(OAc)₂ (5 mol %), PPh₃ (15 mol %), pivalic anhydride (1.5 eq), water (2.5 eq) and THF (3V). The reaction was stirred at ambient temperature for 1 h followed by heating to 60° C. and holding for 24 h. The reaction was cooled to ambient temperature to give a THF solution of the title compound.

### Part B: Preparation of tert-butyl (S)-(1-amino-5-(4-((2-fluorobenzyl)oxy)phenyl)-1,5-dioxopentan-2-yl)carbamate

To the THF solution from Part A was charged NH₄OH solution (5V) with good agitation and the reaction was aged for an additional 36 h at ambient temperature. Following addition of EtOAc (20V) and water (10V), the layers were split, and the organic layer was filtered through a pad of Celite®/silica gel, and the spent pad was washed with EtOAc (20V). The filtrate was concentrated in vacuo. To the resulting residue was added MTBE (30V) and the mixture was heated to reflux and aged for about 15 min. After cooling to ambient temperature, the reaction was further cooled to 0-5° C. and aged for about 15 min. The solids were filtered and twice washed with ice cold MTBE (4V). The resulting solids were slurried in heptane (20V) and the mixture was heated to reflux and aged for about 15 min. After cooling to ambient temperature, the slurry was further cooled to 0-5° C. and aged for about 15 min. The solids were filtered, twice washed with heptane (4V) and dried to constant weight in vacuo to give 5.88 g (83%) of the title compound as a grey solid.

### Example 32: Preparation of (S)-5-(4-((2-fluorobenzyl)oxy)phenyl)-3,4-dihydro-2H-pyrrole-2-car examide

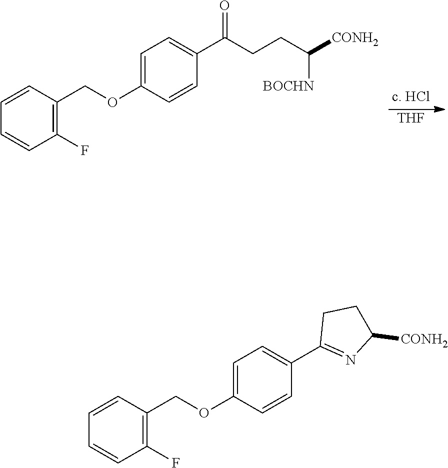

A reactor was charged with of tert-butyl (S)-(1-amino-5-(4-((2-fluorobenzyl)oxy)phenyl)-1,5-dioxopentan-2-yl)carbamate (1.0 g) and THF (10V). After cooling the solution to about 0° C., concentrated HCl (5V) was added, the reaction was warmed to ambient temperature and stirred for 90 min. The reaction was quenched by addition of saturated NaHCO₃ solution, IPA (4V) was charged to the slurry and stirring was continued for about 30 min. Solids were filtered, washed with water (2V) and dried to constant weight in vacuo at 40° C. to give 586 mg (81%) of the title compound.

### Example 33: Preparation of (S)-5-(4-((2-fluorobenzyl)oxy)phenyl)-3,4-dihydro-2H-pyrrole-2-carboxamide

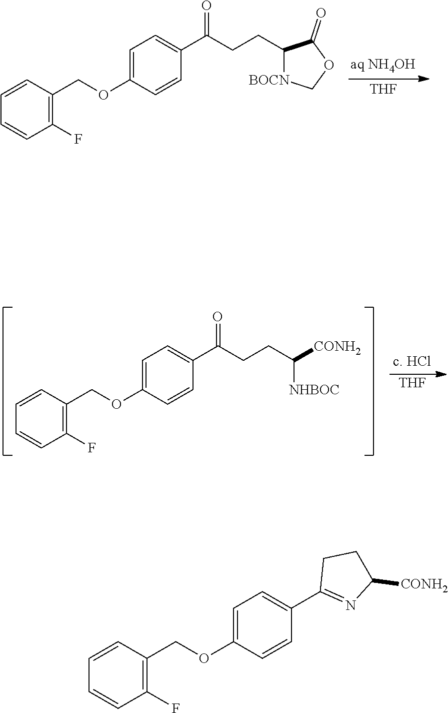

### Part A: In Situ Preparation of tert-butyl (S)-(1-amino-5-(4-((2-fluorobenzyl)oxy)phenyl)-1,5-dioxopentan-2-yl)carbamate

A reactor was charged with tert-butyl (S)-4-(3-(4-((2-fluorobenzyl)oxy)phenyl)-3-oxopropyl)-5-oxooxazolidine-3-carboxylate (100 mg), THF (10V) and NH₄OH solution (10V). The reaction was stirred at ambient temperature for 8 h then heated to and maintained at 60° C. for 3 h. The reaction was cooled to ambient temperature to give a THF solution of the title compound.

### Part B: Preparation of (S)-5-(4-((2-fluorobenzyl)oxy)phenyl)-3,4-dihydro-2H-pyrrole-2-carboxamide

To the solution from Part A was charged THF (10V) and concentrated HCl (10V), and the reaction was stirred at ambient temperature for 6 h. The reaction was quenched by addition of saturated NaHCO₃ solution followed by concentration to half volume at 40° C. in vacuo. The reaction was thrice extracted with EtOAc (10V) and the combined organic layers were concentrated to dryness. The resulting residue was stirred for about 15 min with MTBE (5V), and the solids were filtered and washed with ice cold MTBE (5V). The solids were dried to constant weight in vacuo to give 575 mg (82%) of the title compound as a white solid.

### Example 34: Preparation of (S)-5-(4-((2-fluorobenzyl)oxy)phenyl)-3,4-dihydro-2H-pyrrole-2-carboxamide

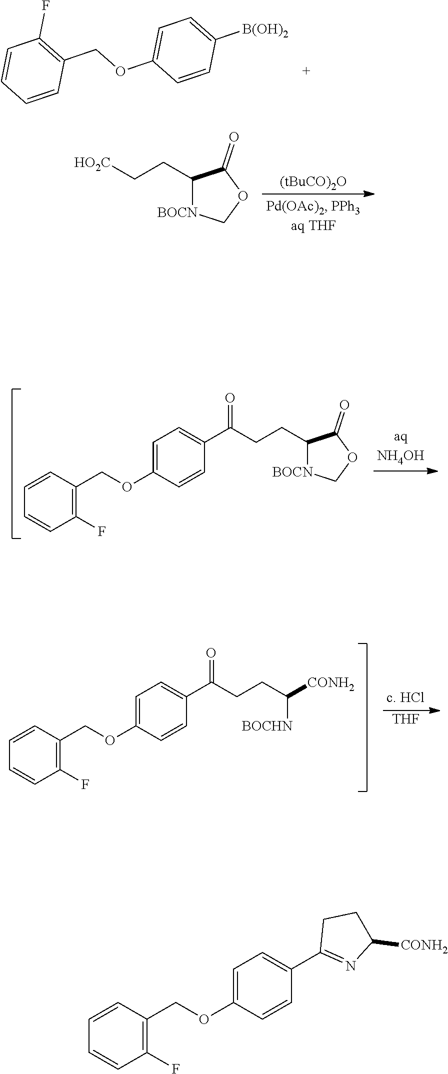

### Part A: In Situ Preparation of tert-butyl (S)-4-(3-(4-((2-fluorobenzyl)oxy)phenyl)-3-oxopropyl)-5-oxooxazolidine-3-carboxylate

A nitrogen flushed 5 L reactor was charged with (S)-3-(3-(tert-butoxycarbonyl)-5-oxooxazolidin-4-yl)propanoic acid (225 g), (4-((2-fluorobenzyl)oxy)phenyl)boronic acid (1.3 eq), PPh₃ (15 mol %), Pd(OAc)₂ (5 mol %) and THF (10V) under an inert atmosphere of nitrogen. After stirring for about 5 min at ambient temperature, pivalic anhydride (1.5 eq) and water (2.5 eq) were charged, and after stirring an additional 5 min, the mixture was sparged with nitrogen for about 15 min. The mixture was aged for about 1 h, heated to 58-60° C. (reaction temperature) over about 1 h and aged for about 24 h. The progress of the reaction was monitored for completion (HPLC). The reaction mixture was cooled to room temperature under an inert atmosphere of nitrogen to give a THF solution of the title compound.

### Part B: In Situ Preparation of tert-butyl (S)-(1-amino-5-(4-((2-fluorobenzyl)oxy)phenyl)-1,5-dioxopentan-2-yl)carbamate

The THF solution from Part A was transferred into a 12 L reactor and was cooled to about 0° C. 28 wt % NH₄OH solution (10.2V) was added with good agitation while maintaining a reaction temperature of <5° C. The resulting mixture was warmed to ambient temperature over about 1 h and was aged for an additional 48 h; the progress of the reaction was monitored for completion (HPLC). The mixture was concentrated in vacuo at 20-25° C. for about 3 h followed by sparging with nitrogen for about 4 h to afford an solution of the title compound.

### Part C: Preparation of (S)-5-(4-((2-fluorobenzyl)oxy)phenyl)-3,4-dihydro-2H-pyrrole-2-carboxamide

To the solution from Part B was added THF (15.6V), the solution was aged for 1 h at ambient temperature then cooled to about 0° C. over about 2 h. 37 wt % HCl (10V) was added with good agitation while maintaining a reaction temperature of <10° C. The mixture was warmed to ambient temperature over about 1 h and aged for about 2.5 h; the progress of the reaction was monitored for completion (HPLC). The solution was divided into two equal portions for work up in parallel.

Each portion was cooled to about 0° C. over about 2 h and water (30.2V) and EtOAc (20V) were charged. After stirring for about 15 min, the layers were separated and the layer was back extracted with EtOAc (20V). The layers from each run were individually cooled to about 0° C. over about 1 h, and the pH was adjusted to ˜8 by addition of 10M aq NaOH solution. After charging EtOAc (20V) to each portion, the mixtures were stirred for about 30 min, the layers were separated and the layers were back extracted with EtOAc (9.8V). After stirring for about 15 min, the layers were separated and all EtOAc layers were combined and treated with aq NaOH (pH 10, 10V) for about 1 h. The layers were separated, the organic layer was passed through a pad of silica gel and Celite® and the spent pad was washed with EtOAc (4.4V). The filtrate was concentrated in vacuo at 50° C. to minimum volume, and the resulting residue was reconcentrated twice from MTBE (4.9V). MTBE (9.8V) was charged, the mixture was heated to reflux, aged about 1 h, cooled to room temperature over about 2 h, cooled to about 0° C. over 2 h and aged for an additional 2 h. The solids were isolated by filtration, washed with ice cold MTBE (2V) and dried in vacuo overnight at ambient temperature. The solids were stirred for about 18 h at ambient temperature after charging MTBE (8.9V) and IPA (1V). The mixture was cooled to about 0° C. over 2 h, filtered and washed with ice cold MTBE (2V). The solids were dried in vacuo overnight to give 62.8 g (23%) of the title compound as a white solid.

### Example 35: Preparation of methyl (2S,5R)-5-(4-((2-fluorobenzyl)oxy)phenyl)pyrrolidine-2-carboxylate

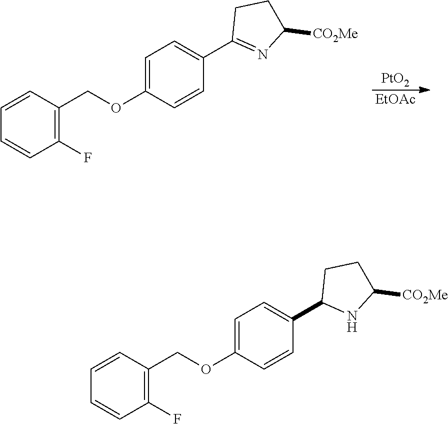

A reactor was charged with methyl (S)-5-(4-((2-fluorobenzyl)oxy)phenyl)-3,4-dihydro-2H-pyrrole-2-carboxylate (100 mg), EtOAc (10V) and PtO₂ (10 wt %) under an inert atmosphere of nitrogen. The mixture was stirred at ambient temperature under 1 atm hydrogen gas pressure; the progress of the reaction was monitored for completion (HPLC). After about 6 h, the mixture was filtered, the spent pad was washed twice with EtOAc (5V) and the filtrate was concentrated in vacuo. The resulting residue was purified by column chromatography to give 87 mg (86%) of the title compound.

### Example 36: Preparation of (2S,5R)-5-(4-((2-fluorobenzyl)oxy)phenyl)pyrrolidine-2-carboxamide hydrochloride (1:1)

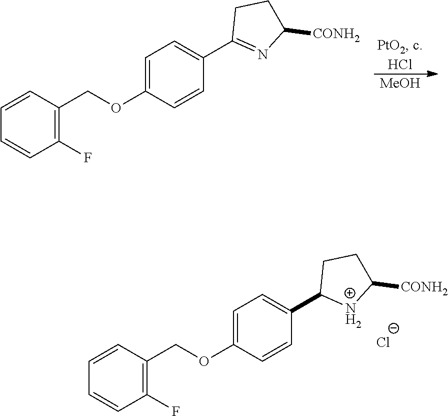

A reactor was charged with (S)-5-(4-((2-fluorobenzyl)oxy)phenyl)-3,4-dihydro-2H-pyrrole-2-carboxamide (62 g), MeOH (10V), 37 wt % HCl (1 eq) and PtO₂ (9.7 wt %) under an inert atmosphere of nitrogen. The mixture was stirred at ambient temperature under 1 atm hydrogen gas pressure; the progress of the reaction was monitored for completion (HPLC). After about 30 h, the mixture was filtered through a pad of Celite®, the spent pad was washed with MeOH (6.5V) and the filtrate was concentrated in vacuo at 35° C. until distillate collection slowed considerably. The resulting residue was reconcentrated in vacuo at 40° C. in triplicate from IPA (4.8V). To the resulting residue was charged IPA (5.8V) and water (1V). The mixture was heated to 70° C., aged about 1 h, cooled to ambient temperature over about 2 h, cooled to 0-5° C. and aged for about 3 h. Solids were isolated by filtration and dried to give 32.2 g (46%) of product. The solids (31.5 g) were dissolved in MeOH (10.2V), decolorizing charcoal (10 wt %) was added and the mixture was stirred at ambient temperature for about 18 h. The mixture was filtered through a Celite® pad, and the spent pad was washed with MeOH (4.1V). The filtrate was concentrated to dryness in vacuo to give 22.4 g (71% recovery) of product. The solids were dissolved in 6:1 (v/v) IPA-water (6.9V) at 70° C., the solution was aged for about 1 h, cooled to ambient temperature over about 2 h, cooled to 0-5° C. and aged for about 3 h. Solids were isolated by filtration, washed with ice cold 6:1 (v/v) IPA-water (1V) and dried to constant weight in vacuo to give 10.23 g (46% recovery) of the title compound.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A process for preparing a compound of formula (I) 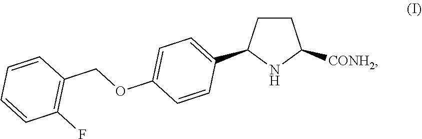 [SMILES: NC(=O)[C@@H]1CC[C@H](c2ccc(OCc3ccccc3F)cc2)N1]
or a pharmaceutically acceptable salt thereof, comprising reacting a compound of formula (II) with a compound of formula (IV) in the presence of a base, such as lithium bis(trimethylsilyl amide), thereby producing a compound of formula (V): 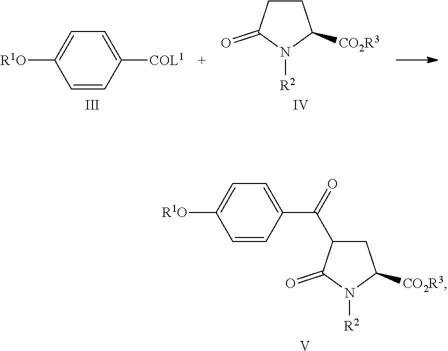
wherein:
L¹ is a leaving group;
R¹ is an oxygen-protecting group;
R² is a resonance-accepting nitrogen-protecting group; and
R³ is C₁₋₆ alkyl, C₂₋₆ alkenyl, or C₂₋₆ alkynyl.

2. The process of claim 1, further comprising reacting a compound of formula (II) with a carboxyl-activating compound, such as oxalyl chloride, thereby producing the compound of formula (II):

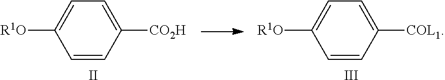

3. The process of claim 2, further comprising deprotecting the compound of formula (V), thereby producing a compound of formula (VI):

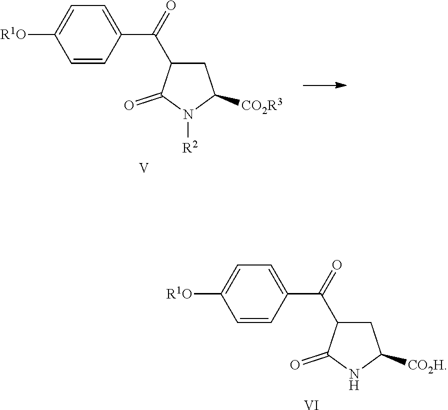

4. A process for preparing a compound of formula (I) 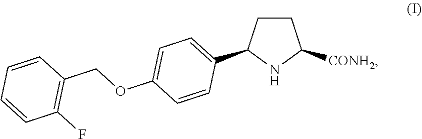 [SMILES: NC(=O)[C@@H]1CC[C@H](c2ccc(OCc3ccccc3F)cc2)N1]
or a pharmaceutically acceptable salt thereof, comprising deprotecting a compound of formula (V), thereby producing a compound of formula (VI): 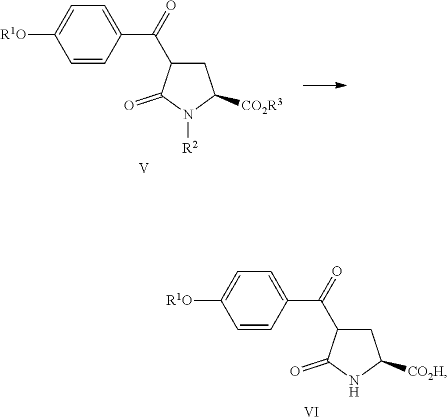
wherein:
R¹ is an oxygen-protecting group;
R² is a resonance-accepting nitrogen-protecting group; and
R³ is C₁₋₆ alkyl, C₂₋₆ alkenyl, or C₂₋₆ alkynyl.

5. The process of claim 3 or 4, wherein deprotecting the compound of formula (V) comprises reacting the compound of formula (V) with an acid, such as hydrochloric acid.

6. The process of any one of claims 3-5, further comprising reacting the compound of formula (VI) with an acid, thereby producing a compound (1), or a salt thereof:

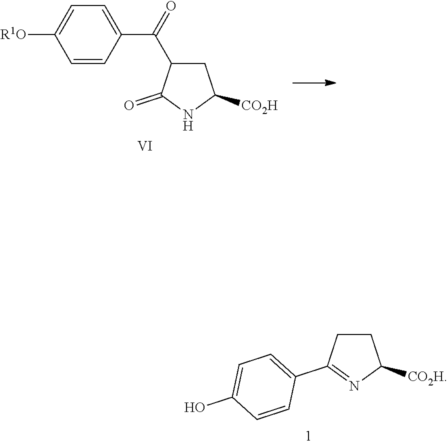

7. A process for preparing a compound of formula (I) 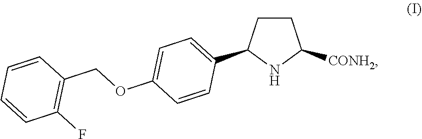 [SMILES: NC(=O)[C@@H]1CC[C@H](c2ccc(OCc3ccccc3F)cc2)N1]
or a pharmaceutically acceptable salt thereof, comprising reacting a compound of formula (VI) with an acid, such as hydrochloric acid, thereby producing a compound (1), or a salt thereof: 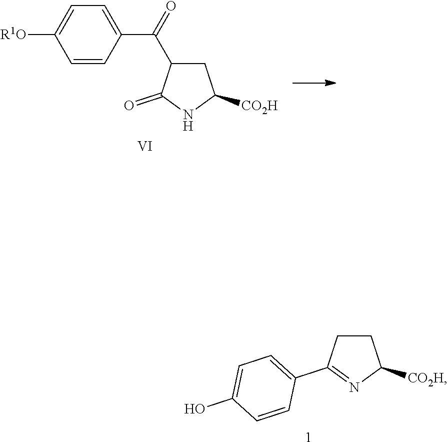
wherein R¹ is an oxygen-protecting group.

8. The process of claim 6 or 7, further comprising reacting the compound (1), or a salt thereof, with methanol, thereby producing a compound (2), or a salt thereof:

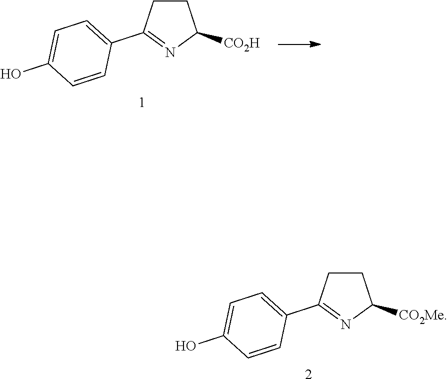

9. The process of claim 8, comprising reacting the compound (1), or a salt thereof, with methanol in the presence of an acid, such as hydrochloric acid.

10. The process of claim 8 or 9, further comprising reacting the compound (2), a salt thereof, with hydrogen gas and a compound that provides a resonance-accepting nitrogen-protecting group, such as di-tert-butyldicarbonate, in the presence of a catalyst, such as Pd(OH)₂/C, thereby producing a compound of formula (VII): 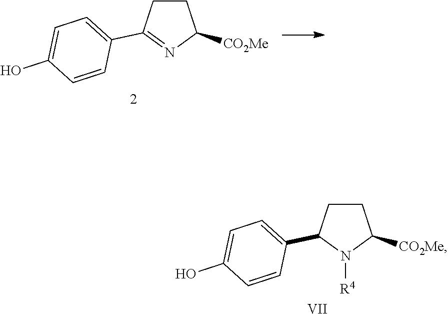
wherein R⁴ is a resonance-accepting nitrogen-protecting group.

11. The process of claim 10, comprising:
reacting a compound of formula (II) with a carboxyl-activating compound, thereby producing a compound of formula (III): 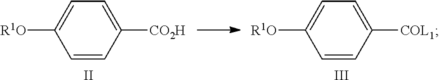
reacting the compound of formula (I) with a compound of formula (IV) in the presence of a base, thereby producing a compound of formula (V): 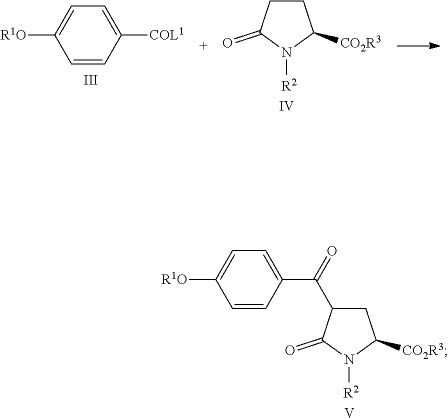
deprotecting the compound of formula (V), thereby producing a compound of formula (VI): 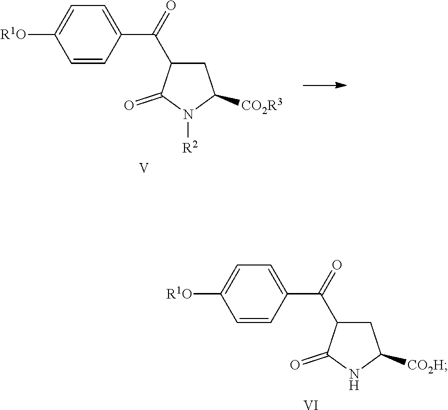
reacting the compound of formula (IV) with an acid, thereby producing a compound (1), or a salt thereof: 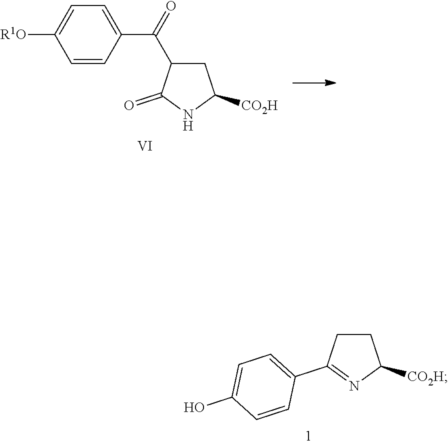
reacting the compound (1) with methanol, thereby producing a compound (2), or a sat thereof: 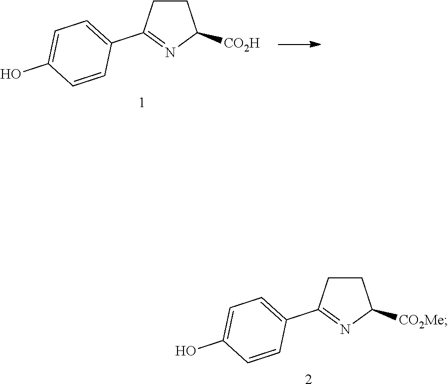
and
reacting the compound (2), or a salt thereof, with hydrogen gas and a compound that provides a resonance-accepting nitrogen-protecting group in the presence of a catalyst, thereby producing the compound of formula (VII):

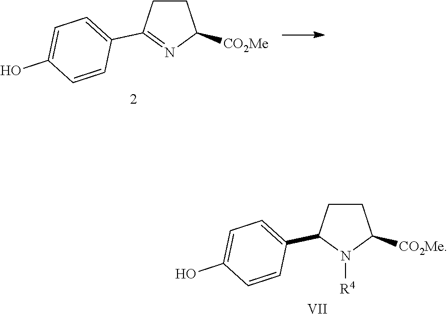

12. A process for preparing a compound of formula (I) 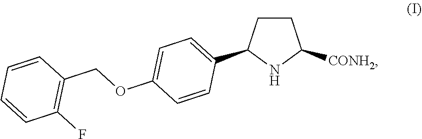 [SMILES: NC(=O)[C@@H]1CC[C@H](c2ccc(OCc3ccccc3F)cc2)N1]
or a pharmaceutically acceptable salt thereof, comprising reacting a compound of formula (VIII) with a compound of formula (IX), thereby producing a compound of formula (X): 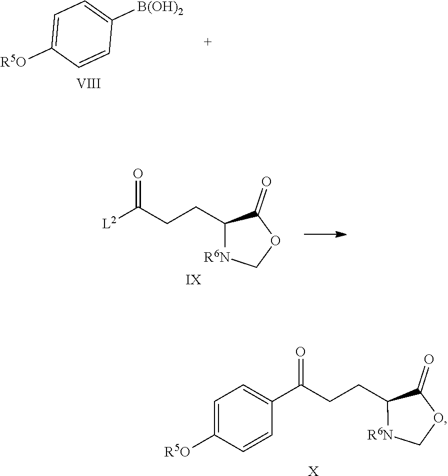
wherein:
L² is hydroxyl or a leaving group;
R⁵ is 2-fluorobenzyl or an oxygen-protecting group; and
R⁶ is a resonance-accepting nitrogen-protecting group.

13. The process of claim 12, comprising reacting the compound of formula (VIII) with the compound of formula (IX) in the presence of a palladium coupling reagent.

14. The process of claim 11 or 12, further comprising reacting the compound of formula (X) with an acid, thereby producing a compound of formula (XI):

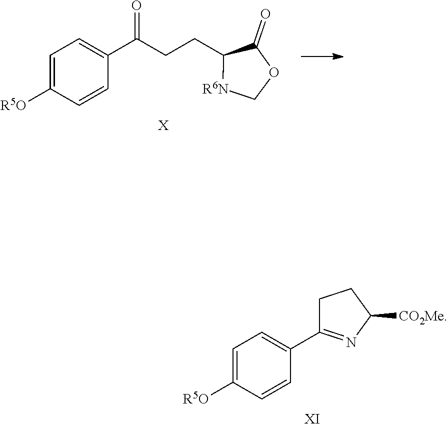

15. A process for preparing a compound of formula (I) 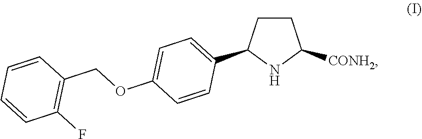 [SMILES: NC(=O)[C@@H]1CC[C@H](c2ccc(OCc3ccccc3F)cc2)N1]
or a pharmaceutically acceptable salt thereof, comprising reacting a compound of formula (X) with an acid, such as methanesulfonic acid and/or sulfuric acid, thereby producing a compound of formula (XI): 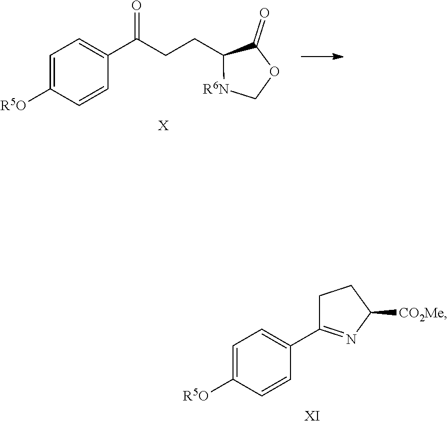
wherein:
R⁵ is 2-fluorobenzyl or an oxygen-protecting group; and
R⁶ is a resonance-accepting nitrogen-protecting group.

16. The process of claim 14 or 15, further comprising reacting the compound of formula (XI) with NH₄OH, thereby producing a compound of formula (XII):

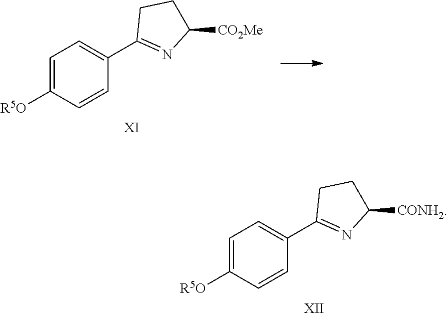

17. A process for preparing a compound of formula (I) 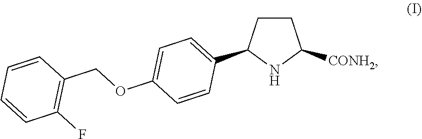 [SMILES: NC(=O)[C@@H]1CC[C@H](c2ccc(OCc3ccccc3F)cc2)N1]
or a pharmaceutically acceptable salt thereof, comprising reacting a compound of formula (XI) with NH₄OH, thereby producing a compound of formula (XII): 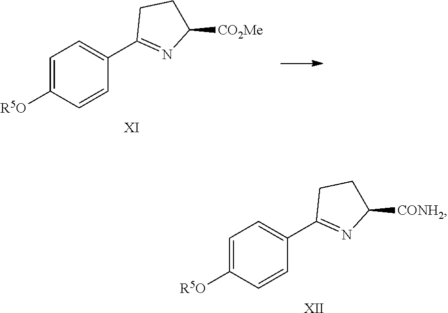
wherein R⁵ is 2-fluorobenzyl or an oxygen-protecting group.

18. A process for preparing a compound of formula (I) 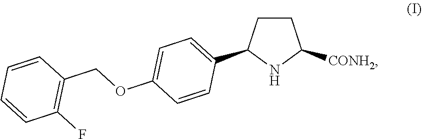 [SMILES: NC(=O)[C@@H]1CC[C@H](c2ccc(OCc3ccccc3F)cc2)N1]
or a pharmaceutically acceptable salt thereof, comprising reacting a compound of formula (XXII) with a compound of formula (VIII), thereby producing a compound of formula (XXIII): 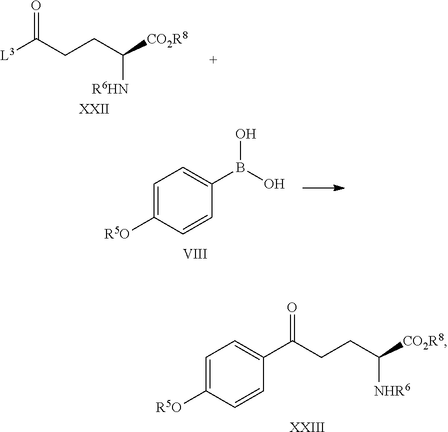
wherein:
L¹ is a leaving group;
R⁵ is 2-fluorobenzyl or an oxygen-protecting group;
R⁶ is a resonance-accepting nitrogen-protecting group; and
R⁸ is C₁₋₆alkyl, C₂₋₆ alkenyl, or C₂₋₆ alkynyl.

19. The process of claim 18, comprising reacting the compound of formula (XXII) with the compound of formula (VIII) in the presence of a palladium coupling reagent.

20. The process of claim 18 or 19, further comprising reacting the compound of formula (XXIII) with NH₄OH, thereby producing a compound of formula (XIII)

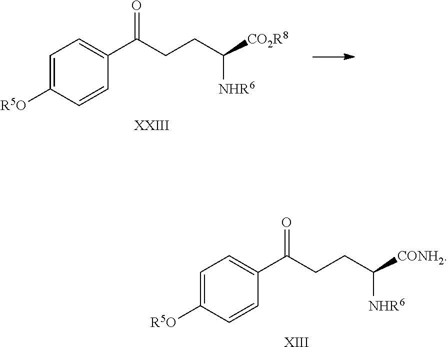

21. A process for preparing a compound of formula (I) 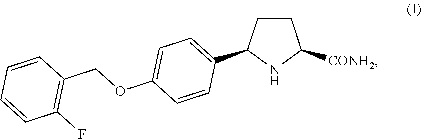 [SMILES: NC(=O)[C@@H]1CC[C@H](c2ccc(OCc3ccccc3F)cc2)N1]
or a pharmaceutically acceptable salt thereof, comprising reacting a compound of formula (XXI) with NH₄OH, thereby producing a compound of formula (XII): 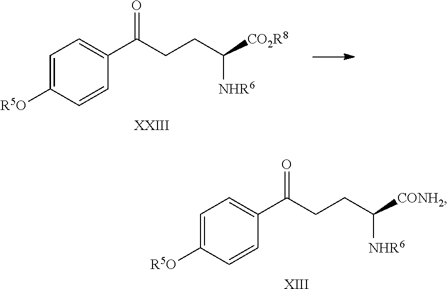
wherein:
R⁵ is 2-fluorobenzyl or an oxygen-protecting group;
R⁶ is a resonance-accepting nitrogen-protecting group; and
R⁸ is C₁₋₆ alkyl, C₂₋₆ alkenyl, or C₂₋₆ alkynyl.

22. A process for preparing a compound of formula (I) 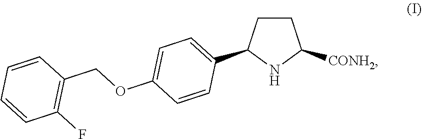 [SMILES: NC(=O)[C@@H]1CC[C@H](c2ccc(OCc3ccccc3F)cc2)N1]
or a pharmaceutically acceptable salt thereof, comprising reacting a compound of formula (X) with NH₄OH, thereby producing a compound of formula (XIII): 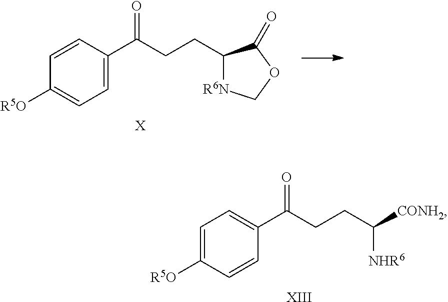
wherein:
R⁵ is 2-fluorobenzyl or an oxygen-protecting group; and
R⁶ is a resonance-accepting nitrogen-protecting group.

23. The process of any one of claims 20-22, further comprising deprotecting the compound of formula (XIII), thereby producing a compound of formula (XII):

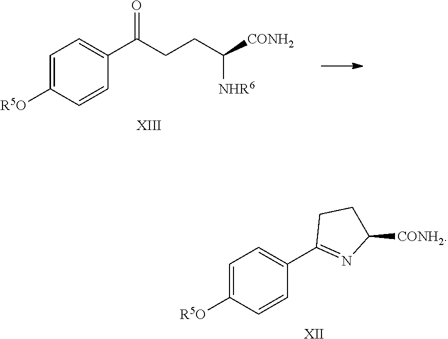

24. A process for preparing a compound of formula (I) 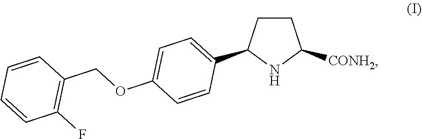 [SMILES: NC(=O)[C@@H]1CC[C@H](c2ccc(OCc3ccccc3F)cc2)N1]
or a pharmaceutically acceptable salt thereof, comprising deprotecting a compound of formula (XIII), thereby producing a compound of formula (XII): 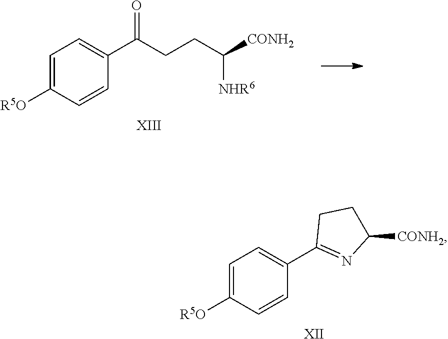
wherein:
R⁵ is 2-fluorobenzyl or an oxygen-protecting group; and
R⁶ is a resonance-accepting nitrogen-protecting group.

25. The process of claim 23 or 24, further comprising reacting the compound of formula (XII) with hydrogen gas in the presence of a catalyst, thereby producing a compound of formula (XIV):

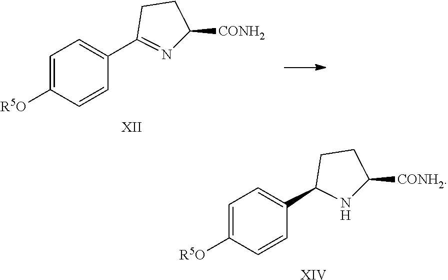

26. A process for preparing a compound of formula (I) 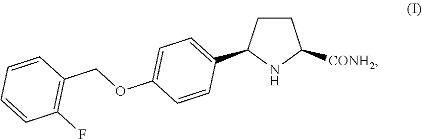 [SMILES: NC(=O)[C@@H]1CC[C@H](c2ccc(OCc3ccccc3F)cc2)N1]
or a pharmaceutically acceptable salt thereof, comprising reacting a compound of formula (XII) with hydrogen gas in the presence of a catalyst, such as PtO₂, thereby producing a compound of wherein R⁵ is 2-fluorobenzyl or an oxygen-protecting group.

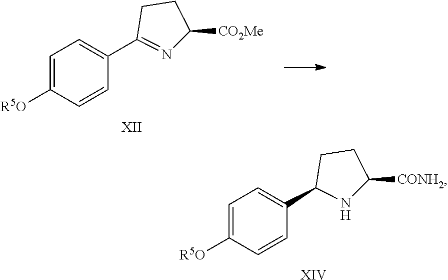

27. The process of claim 25 or 26, comprising reacting the compound of formula (XII) with hydrogen gas in the presence of a catalyst, such as PtO₂ and a solvent, such as methanol.

28. The process of any one of claims 25-27, further comprising reacting the compound of formula (XIV) with hydrochloric acid, thereby producing a compound of formula (XV):

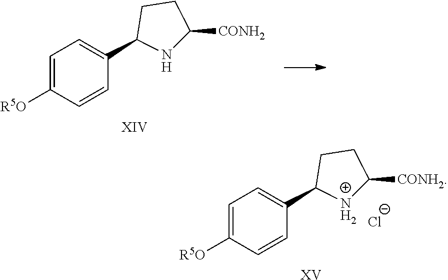

29. The process of any one of claims 12-28, wherein R⁵ is 2-fluorobenzyl or an oxygen-protecting group selected from benzyl, benzoyl, methoxymethyl, tetrahydropyranyl, tert-butyl, acetyl, and silicon-containing protecting group.

30. The process of claim 29, wherein R⁵ is 2-fluorobenzyl.

31. The process of any one of claims 12-24, further comprising reacting the compound of formula (XII) with hydrogen gas in the presence of a catalyst, thereby producing a compound of formula (XVI):

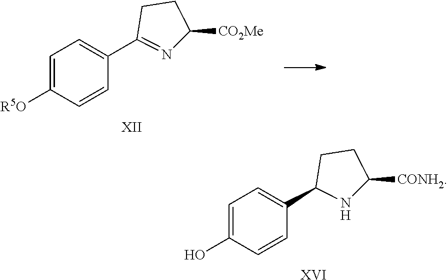

32. A process for preparing a compound of formula (I) 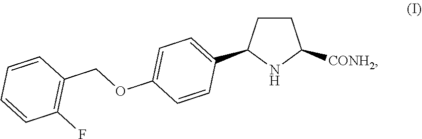 [SMILES: NC(=O)[C@@H]1CC[C@H](c2ccc(OCc3ccccc3F)cc2)N1]
or a pharmaceutically acceptable salt thereof, comprising reacting a compound of formula (XII) with hydrogen gas in the presence of a catalyst, thereby producing a compound of formula (XVI): 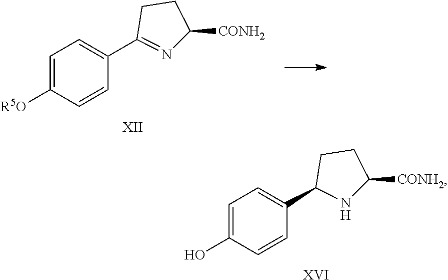
wherein R⁵ is 2-fluorobenzyl or an oxygen-protecting group.

33. The process of claim 31 or 32, further comprising reacting the compound of formula (XII) with hydrogen gas in the presence of a catalyst, such as Pd(OH)₂/C, preferrably 20 wt % Pd(OH)₂/C and a solvent, such as tetrahydrofuran or methanol.

34. The process of any one of claims 31-33, wherein R⁵ is an oxygen-protecting group selected from benzyl, benzoyl, methoxymethyl, tetrahydropyranyl, tert-butyl, acetyl, and silicon-containing protecting group.

35. The process of any one of claims 80-85, wherein R⁵ is benzyl.

36. The process of any one of claims 31-35, further comprising reacting the compound of formula (XVI) with a compound that provides a resonance-accepting nitrogen-protecting group, thereby producing a compound of formula (XVII): 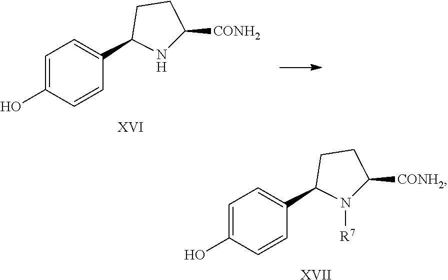
wherein R⁷ is a resonance-accepting nitrogen-protecting group.

37. The process of claim 36, further comprising reacting the compound of formula (XVI) with the compound that provides a resonance-accepting nitrogen-protecting group in the presence of a solvent, such as tetrahydrofuran or methanol.

38. The process of claim 36 or 37, wherein the compound that provides a resonance-accepting nitrogen-protecting group is di-tert-butyldicarbonate.

39. The process of any one of claims 36-38, further comprising reacting the compound of formula (XVII) with a compound of formula (XVIII), thereby producing a compound of formula 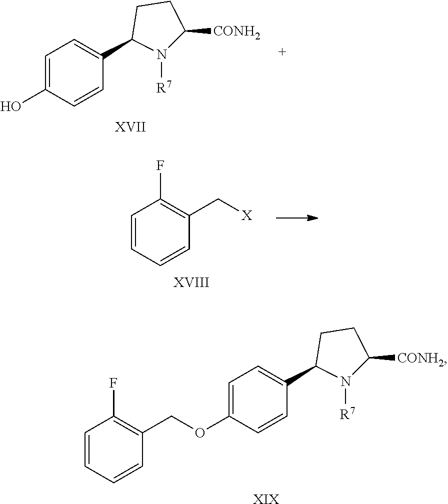
wherein X is a halogen.

40. The process of claim 39, comprising reacting the compound of formula (XVII) with the compound of formula (XVIII) in the presence of a base.

41. The process of claim 39 or 40, comprising reacting the compound of formula (XVII) with the compound of formula (XVIII) in the presence of a base, such as sodium methoxide and a solvent, such as formamide or dimethylformamide.

42. The process of any one of claims 39-31, further comprising deprotecting the compound of formula (XIX), thereby producing a compound of formula (XX):

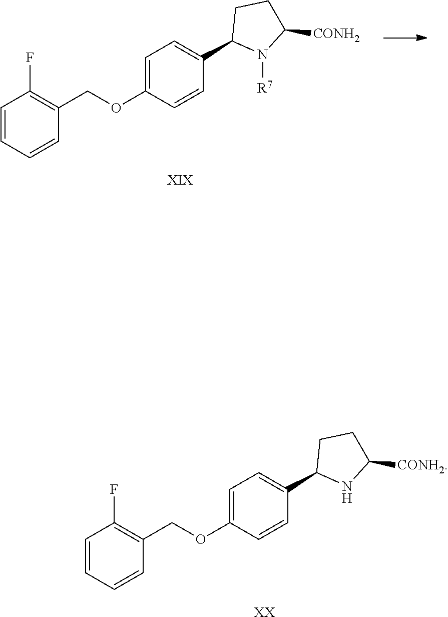

43. The process of claim 42, comprising deprotecting the compound of formula (XIX) in the presence of an acid, such as hydrochloric acid, methanesulfonic acid, or sulfuric acid.

44. The process of claim 42 or 43, comprising deprotecting the compound of formula (XIX) in the presence of an acid, such as hydrochloric acid, methanesulfonic acid or sulfuric acid and a solvent, such as tetrahydrofuran, acetonitrile or methanol.

45. The process of any one of claims 42-44, further comprising reacting the compound of formula (XX) with hydrochloric acid, thereby producing a compound of formula (XXI):

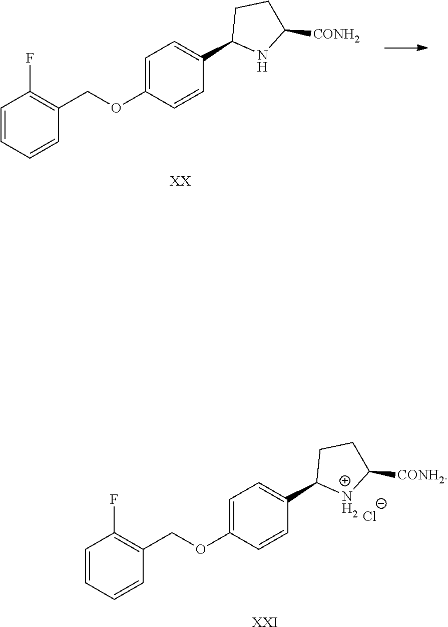

46. The process of claim 45, comprising reacting the compound of formula (XX) with hydrochloric acid in the presence of a solvent, such as tetrahydrofuran, isopropanol or methanol.

47. The process of any one of claims 28-30, comprising:
reacting a compound of formula (VIII) with a compound of formula (IX), thereby producing a compound of formula (X): 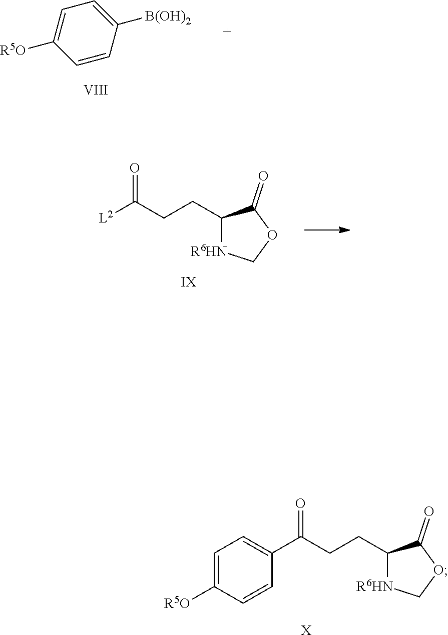
reacting the compound of formula (X) with an acid, thereby producing a compound of formula (X): 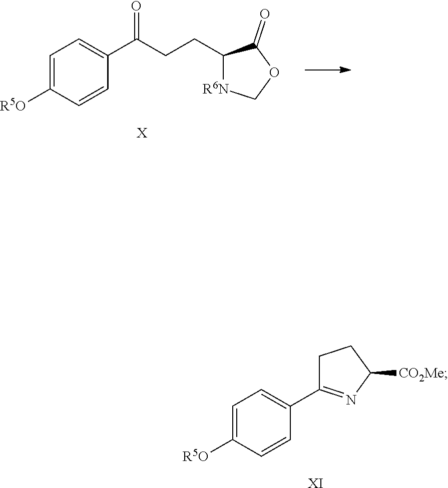
reacting the compound of formula (XI) with NH₄OH, thereby producing a compound of formula (XII): 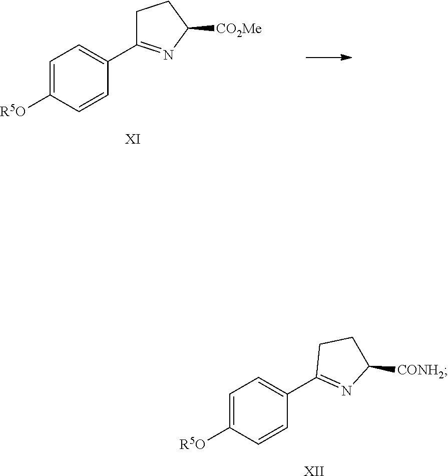
reacting the compound of formula (XII) with hydrogen gas in the presence of a catalyst, thereby producing a compound of formula (XIV): 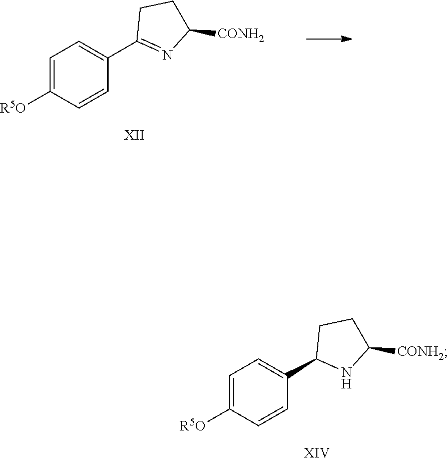
and
reacting the compound of formula (XIV) with hydrochloric acid, thereby producing a compound of formula (XV):

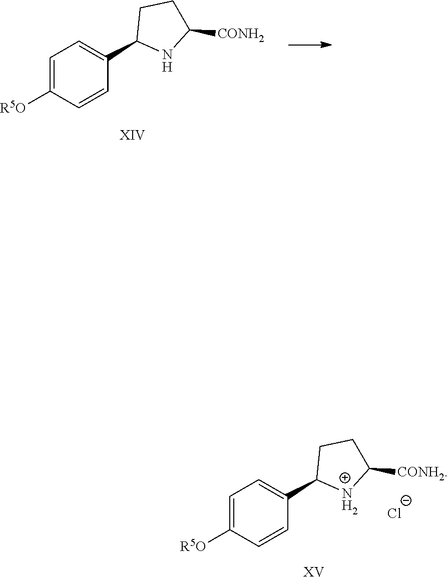

48. The process of claim 45 or 46, comprising:
reacting a compound of formula (XXI) with a compound of formula (VIII), thereby producing a compound of formula (XXII): 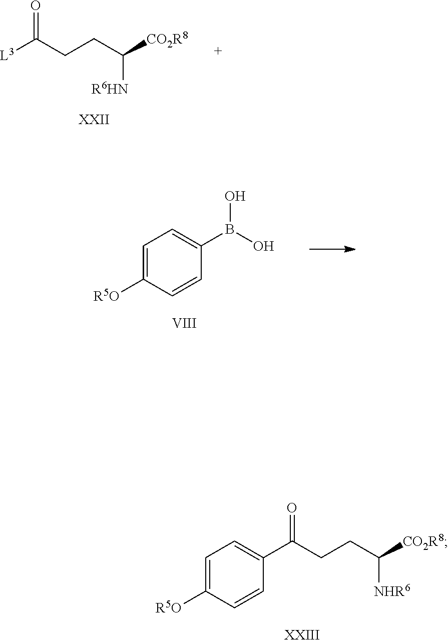
reacting the compound of formula (X) with NH₄OH, thereby producing a compound of formula 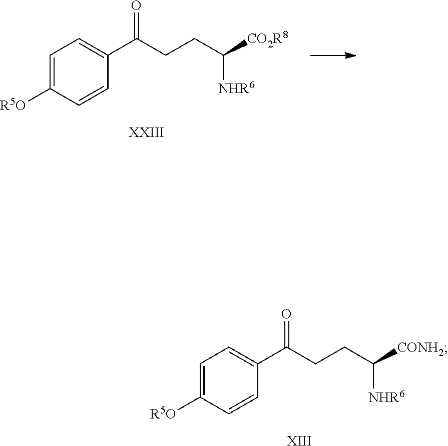
deprotecting the compound of formula (XII), thereby producing a compound of formula (XII): 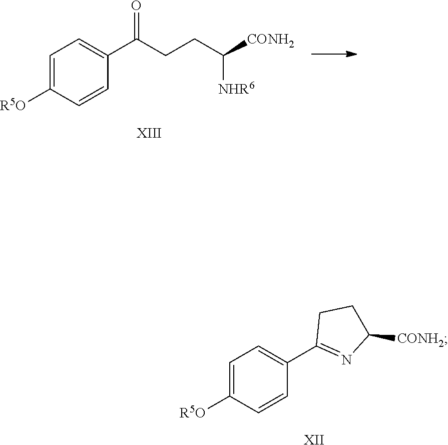
reacting the compound of formula (XII) with hydrogen gas in the presence of a catalyst, thereby producing a compound of formula (XVI): 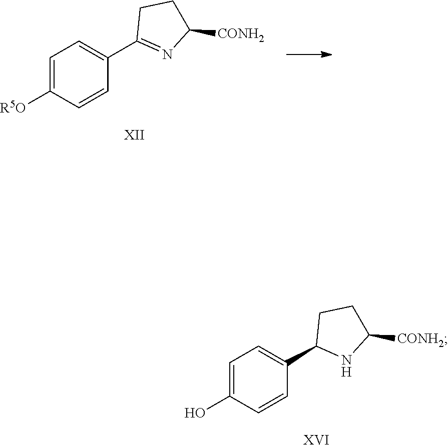
reacting the compound of formula (XVI) with a compound that provides a resonance-accepting nitrogen-protecting group, thereby producing a compound of formula (XVII): 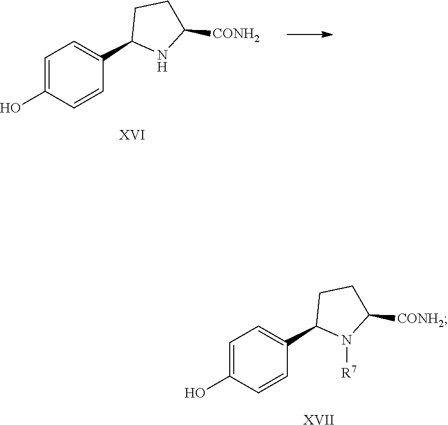
reacting the compound of formula (XVI) with a compound of formula (XVIII), thereby producing a compound of formula (XIX): 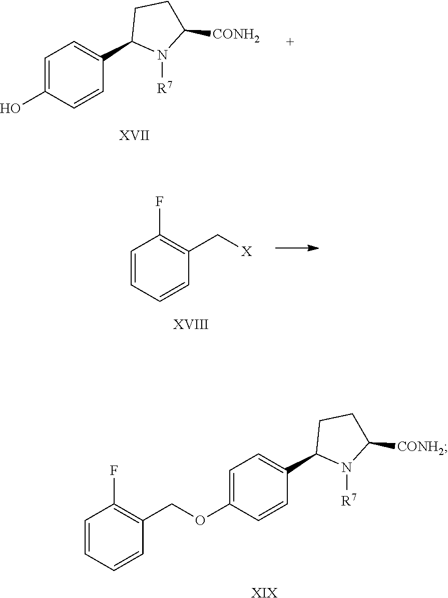
deprotecting the compound of formula (XIX), thereby producing a compound of formula (XX): 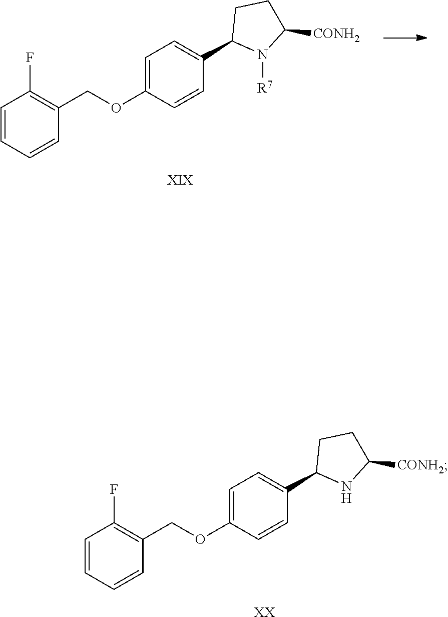
and
reacting the compound of formula (XX) with hydrochloric acid, thereby producing a compound of formula XXI):

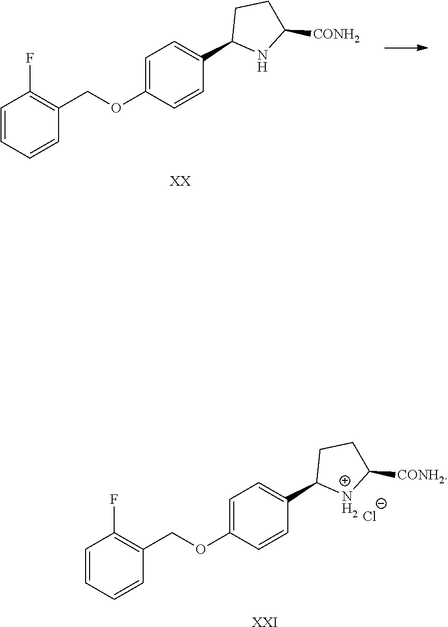

49. A compound of formula (XXIV), formula (XXV), formula (XXVI), formula (XXVII), or formula (XXVIII) 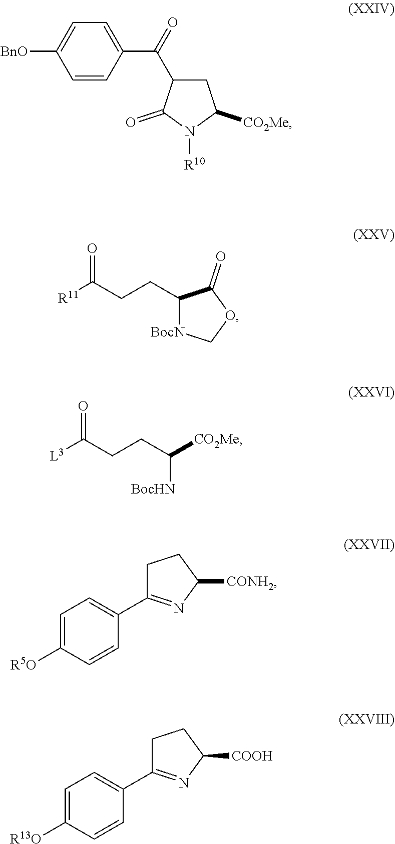
or a pharmaceutically acceptable salt thereof, wherein
R¹⁰ is hydrogen or a resonance-accepting nitrogen-protecting group;
R¹¹ is 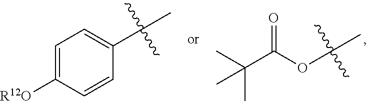
wherein R¹² is 2-fluorobenzyl, benzyl or hydroxyl;
L³ is a leaving group;
R⁵ is benzyl, 2-fluorobenzyl, or an oxygen-protecting group; and
R¹³ is hydrogen, benzyl, 2-fluorobenzyl, or an oxygen-protecting group.

50. The compound of claim 49, having an one of the structures: 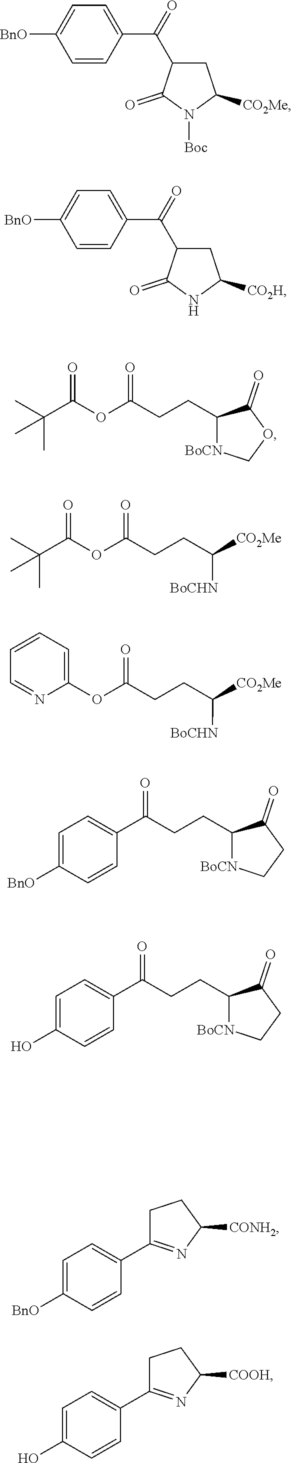 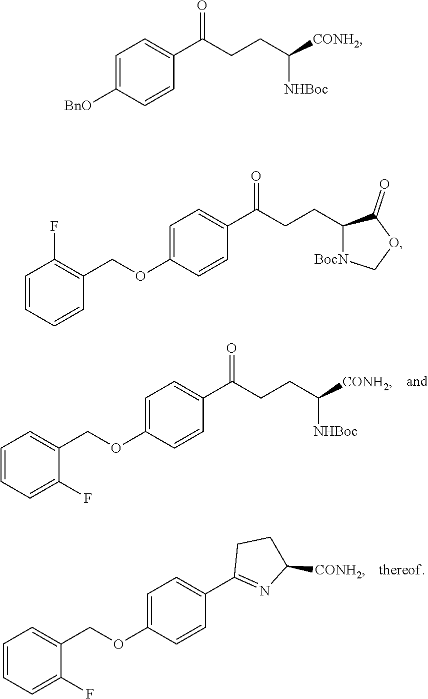 [SMILES: CC(C)(C)OC(=O)N[C@@H](CCC(=O)c1ccc(OCc2ccccc2)cc1)C(N)=O ; CC(C)(C)OC(=O)N1COC(=O)[C@@H]1CCC(=O)c1ccc(OCc2ccccc2F)cc1 ; CC(C)(C)OC(=O)N[C@@H](CCC(=O)c1ccc(OCc2ccccc2F)cc1)C(N)=O ; NC(=O)[C@@H]1CCC(c2ccc(OCc3ccccc3F)cc2)=N1]
or a salt
